(19) United States
(12) Patent Application Publication
(10) Pub. No.: US 2022/0031670 A1
(43) Pub. Date: Feb. 3, 2022

(54) PHARMACEUTICAL COMBINATIONS FOR USE IN THE TREATMENT OF NEOPLASTIC DISEASES

(71) Applicant: Basilea Pharmaceutica International AG, Basel (CH)

(72) Inventors: Felix BACHMANN, Basel (CH); Heidi LANE, Basel (CH); Paul MCSHEEHY, Basel (CH)

(21) Appl. No.: 17/275,727

(22) PCT Filed: Sep. 19, 2019
(86) PCT No.: PCT/EP2019/075177

§ 371 Date: Mar. 12, 2021

(30) Foreign Application Priority Data
Sep. 20, 2018 (EP) 18195748.1

(51) Int. Cl. A61K 31/4245; A61K 31/7068; A61P 35/00

(52) U.S. Cl. CPC A61K 31/4245; A61P 35/00; A61K 31/7068

(57) ABSTRACT

The present invention provides pharmaceutical combinations comprising (a) a compound of formula (I) wherein R represents phenyl or pyridinyl; wherein phenyl is optionally substituted by one or two substituents independently selected from lower alkyl, lower alkoxy, hydroxyl, amino, lower alkylamino, lower dialkylamino, acetylamino, halogen and nitro; and wherein pyridinyl is optionally substituted by amino or halogen; R1 represents hydrogen or cyano-lower alkyl; and wherein the prefix lower denotes a radical having up to and including a maximum of 4 carbon atoms; or a pharmaceutically acceptable derivative thereof; and (b) a compound of Formula (II) (gemcitabine) or a pharmaceutically acceptable salt thereof and the use of the pharmaceutical combinations in the treatment of neoplastic diseases.

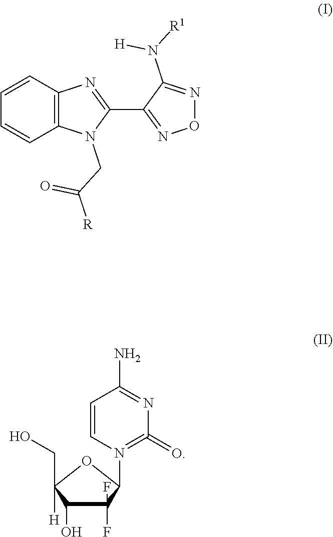

The present invention relates to pharmaceutical combinations comprising two active pharmaceutical compounds as described herein and methods of using the combinations of the invention in the treatment of neoplastic diseases, in particular cancer.

Microtubules are one of the components of the cell cytoskeleton and are composed of heterodimers of alpha and beta tubulin. Agents that target microtubules are among the most effective cytotoxic chemotherapeutic agents and have a broad spectrum of activity. Microtubule destabilising agents (e.g. the vinca-alkaloids such as vincristine, vinblastine and vinorelbine) are used for example in the treatment of several types of hematologic malignancies, such as lymphoblastic leukaemia and lymphoma, as well as solid tumours, such as lung cancer. Microtubule stabilising agents (e.g. the taxanes such as paclitaxel, docetaxel) are used for example in the treatment of solid tumours, including breast, lung and prostate cancer.

WO2004/103994 describes a recently discovered class of microtubule destabilising agents. One compound falling within this class, known as BAL27862 (referred to herein as the compound of formula I-A), and shown in WO2004/103994 under Example 58, has the structure and chemical name given below:

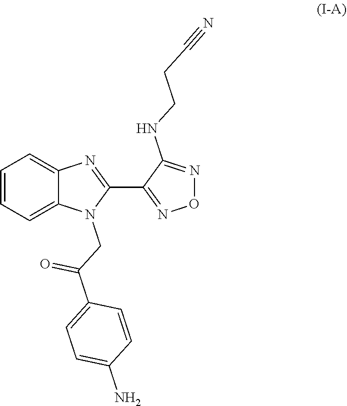 [SMILES: N#CCCNc1nonc1-c1nc2ccccc2n1CC(=O)c1ccc(N)cc1]

### 3-(4-{1-[2-(4-Amino-phenyl)-2-oxo-ethyl]-1H-benzoimidazol-2-yl}-furazan-3-ylamino)-propionitrile

WO2011/012577 discloses pro-drugs of the compounds disclosed in WO2004/103994. One compound known as BAL101553 (referred to herein as the compound of formula I-B) and shown in WO2011/012577 under Example 1 has the chemical name and structure given below:

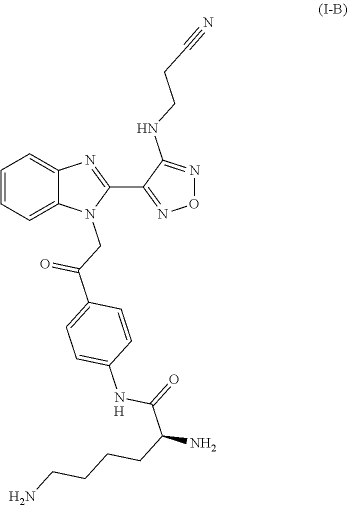

### (S)-2,6-Diamino-hexanoic acid [4-(2-{2-[4-(2-cyano-ethylamino)-furazan-3-yl]-benzoimidazol-1-yl}-acetyl)-phenyl]-amide

The compound of formula I-B is a highly water-soluble pro-drug of the compound of formula I-A which forms the compound of formula I-A following administration. The compound of formula I-B is particularly advantageously used in the form of a pharmaceutically acceptable acid addition salt, such as a hydrochloride salt, in particular in the form of its dihydrochloride salt.

These compounds have been shown to arrest tumour cell proliferation and induce apoptosis. The compound of formula I-A and the pro-drug of formula I-B have demonstrated antitumor activity across a broad panel of experimental tumour models.

Gemcitabine is available commercially as the hydrochloride salt and is marketed e.g. as Gemzar®.

There is an ongoing need for new effective treatment options for cancer patients. As demonstrated in the Examples below it has now surprisingly been found that combinations of the two compounds described above, namely the compound of formula I-A/I-B and gemcitabine provide positive outcomes in cancer models, including cures.

### SUMMARY OF THE INVENTION

In a first aspect the present invention provides a pharmaceutical combination comprising (a) a compound of formula I

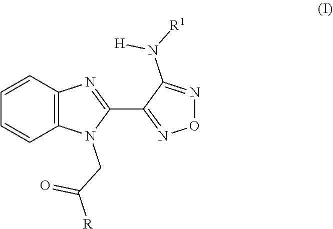

wherein

R represents phenyl or pyridinyl;

wherein phenyl is optionally substituted by one or two substituents independently selected from lower alkyl, lower alkoxy, hydroxyl, amino, lower alkylamino, lower dialkylamino, acetylamino, halogen and nitro;

and wherein pyridinyl is optionally substituted by amino or halogen;

R1 represents hydrogen or cyano-lower alkyl;

and wherein the prefix lower denotes a radical having up to and including a maximum of 4 carbon atoms;

or a pharmaceutically acceptable derivative thereof;

and (b) a compound of formula II (gemcitabine)

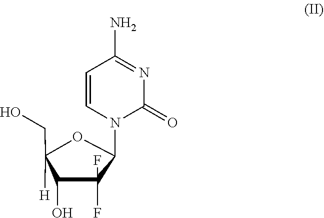

or a pharmaceutically acceptable salt thereof, e.g. the hydrochloride salt

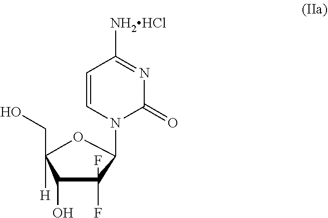

In some embodiment component (a) is a compound of formula I-A or a pharmaceutically acceptable derivative thereof. In further embodiments component (a) is a compound of formula I-A or a pharmaceutically acceptable derivative thereof and component (b) is gemcitabine or a pharmaceutically acceptable salt thereof. In further embodiments component (a) is a compound of formula I-A or a pharmaceutically acceptable salt thereof or a compound of formula I-B or a pharmaceutically acceptable salt thereof. In further embodiments component (a) is a compound of formula I-A or a pharmaceutically acceptable salt thereof or a compound of formula I-B or a pharmaceutically acceptable salt thereof and component (b) is gemcitabine or a pharmaceutically acceptable salt thereof. In further embodiments component (a) is the dihydrochloride salt of the compound of formula I-B. In further embodiments component (a) is the dihydrochloride salt of the compound of formula I-B and component (b) is the hydrochloride salt of gemcitabine.

In a further aspect the invention provides a method for treating a neoplastic disease in a subject in need thereof comprising administering to the subject a therapeutically effective amount of the pharmaceutical combination of the invention. In some embodiments component (a) is a compound of formula I-A or a pharmaceutically acceptable derivative thereof. In further embodiments component (a) is a compound of formula I-A or a pharmaceutically acceptable derivative thereof and component (b) is gemcitabine or a pharmaceutically acceptable salt thereof. In further embodiments component (a) is a compound of formula I-A or a pharmaceutically acceptable salt thereof or a compound of formula I-B or a pharmaceutically acceptable salt thereof. In further embodiments component (a) is a compound of formula I-A or a pharmaceutically acceptable salt thereof or a compound of formula I-B or a pharmaceutically acceptable salt thereof and component (b) is gemcitabine or a pharmaceutically acceptable salt thereof. In further embodiments component (a) is the dihydrochloride salt of the compound of formula I-B. In further embodiments component (a) is the dihydrochloride salt of the compound of formula I-B and component (b) is the hydrochloride salt of gemcitabine.

In a further aspect the invention provides a method for treating a neoplastic disease in a subject in need thereof comprising administering to the subject a therapeutically effective amount of the compound of formula I or a pharmaceutically acceptable derivative thereof, wherein said subject is undergoing or will undergo treatment with the compound of formula II or pharmaceutically acceptable salt thereof. In some embodiments the compound of formula I or pharmaceutically acceptable derivative thereof is a compound of formula I-A or a pharmaceutically acceptable derivative thereof. In further embodiments the compound of formula I or pharmaceutically acceptable derivative thereof is a compound of formula I-A or a pharmaceutically acceptable derivative thereof and the compound of formula II or pharmaceutically acceptable salt thereof is gemcitabine or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof and the compound of formula II or pharmaceutically acceptable salt thereof is gemcitabine or a pharmaceutically acceptable salt thereof. In further embodiments the compound of formula I or pharmaceutically acceptable derivative thereof is the dihydrochloride salt of the compound of formula I-B. In further embodiments the compound of formula I or pharmaceutically acceptable derivative thereof is the dihydrochloride salt of the compound of formula I-B and the compound of formula II or pharmaceutically acceptable salt thereof is the hydrochloride salt of gemcitabine.

In a further aspect the invention provides a method for treating a neoplastic disease in a subject in need thereof comprising administering to the subject a therapeutically effective amount of the compound of formula II or a pharmaceutically acceptable salt thereof, wherein said subject is undergoing or will undergo treatment with the compound of formula I or pharmaceutically acceptable derivative thereof. In some embodiments the compound of formula I or pharmaceutically acceptable derivative thereof is a compound of formula I-A or a pharmaceutically acceptable derivative thereof. In further embodiments the compound of formula I or pharmaceutically acceptable derivative thereof is a compound of formula I-A or a pharmaceutically acceptable derivative thereof and the compound of formula II or pharmaceutically acceptable salt thereof is gemcitabine or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof and the compound of formula II or pharmaceutically acceptable salt thereof is gemcitabine or a pharmaceutically acceptable salt thereof. In further embodiments the compound of formula I or pharmaceutically acceptable derivative thereof is the dihydrochloride salt of the compound of formula I-B. In further embodiments the compound of formula I or pharmaceutically acceptable derivative thereof is the dihydrochloride salt of the compound of formula I-B and the compound of formula II or pharmaceutically acceptable salt thereof is the hydrochloride salt of gemcitabine.

In a further aspect the invention provides the pharmaceutical combination of the invention for use in the treatment of a neoplastic disease. In some embodiments component (a) is a compound of formula I-A or a pharmaceutically acceptable derivative thereof. In further embodiments component (a) is a compound of formula I-A or a pharmaceutically acceptable derivative thereof and component (b) is gemcitabine or a pharmaceutically acceptable salt thereof. In further embodiments component (a) is a compound of formula I-A or a pharmaceutically acceptable salt thereof or a compound of formula I-B or a pharmaceutically acceptable salt thereof. In further embodiments component (a) is a compound of formula I-A or a pharmaceutically acceptable salt thereof or a compound of formula I-B or a pharmaceutically acceptable salt thereof and component (b) is gemcitabine or a pharmaceutically acceptable salt thereof. In further embodiments component (a) is the dihydrochloride salt of the compound of formula I-B. In further embodiments component (a) is the dihydrochloride salt of the compound of formula I-B and component (b) is the hydrochloride salt of gemcitabine.

In a further aspect the invention provides a compound of formula I or a pharmaceutically acceptable derivative thereof for use in combination with a compound of formula II or pharmaceutically acceptable salt thereof for the treatment of a neoplastic disease. In some embodiments the compound of formula I or pharmaceutically acceptable derivative thereof is a compound of formula I-A or a pharmaceutically acceptable derivative thereof. In further embodiments the compound of formula I or pharmaceutically acceptable derivative thereof is a compound of formula I-A or a pharmaceutically acceptable derivative thereof and the compound of formula II or pharmaceutically acceptable salt thereof is gemcitabine or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof and the compound of formula II or pharmaceutically acceptable salt thereof is gemcitabine or a pharmaceutically acceptable salt thereof. In further embodiments the compound of formula I or pharmaceutically acceptable derivative thereof is the dihydrochloride salt of the compound of formula I-B. In further embodiments the compound of formula I or pharmaceutically acceptable derivative thereof is the dihydrochloride salt of the compound of formula I-B and the compound of formula II or pharmaceutically acceptable salt thereof is the hydrochloride salt of gemcitabine.

In a further aspect the invention provides a compound of formula II or a pharmaceutically acceptable salt thereof for use in combination with a compound of formula I or pharmaceutically acceptable derivative thereof for the treatment of a neoplastic disease. In some embodiments the compound of formula I or pharmaceutically acceptable derivative thereof is a compound of formula I-A or a pharmaceutically acceptable derivative thereof. In further embodiments the compound of formula I or pharmaceutically acceptable derivative thereof is a compound of formula I-A or a pharmaceutically acceptable derivative thereof and the compound of formula II or pharmaceutically acceptable salt thereof is gemcitabine or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof and the compound of formula II or pharmaceutically acceptable salt thereof is gemcitabine or a pharmaceutically acceptable salt thereof. In further embodiments the compound of formula I or pharmaceutically acceptable derivative thereof is the dihydrochloride salt of the compound of formula I-B. In further embodiments the compound of formula I or pharmaceutically acceptable derivative thereof is the dihydrochloride salt of the compound of formula I-B and the compound of formula II or pharmaceutically acceptable salt thereof is the hydrochloride salt of gemcitabine.

In a further aspect the invention provides use of the pharmaceutical combination of the invention in the preparation of single-agent medicaments or as a combined medicament for the treatment of a neoplastic disease. In some embodiments component (a) is a compound of formula I-A or a pharmaceutically acceptable derivative thereof. In further embodiments component (a) is a compound of formula I-A or a pharmaceutically acceptable derivative thereof and component (b) is gemcitabine or a pharmaceutically acceptable salt thereof. In further embodiments component (a) is a compound of formula I-A or a pharmaceutically acceptable salt thereof or a compound of formula I-B or a pharmaceutically acceptable salt thereof. In further embodiments component (a) is a compound of formula I-A or a pharmaceutically acceptable salt thereof or a gemcitabine or a pharmaceutically acceptable salt thereof mesylate. In further embodiments component (a) is the dihydrochloride salt of the compound of formula I-B. In further embodiments component (a) is the dihydrochloride salt of the compound of formula I-B and component (b) is the hydrochloride salt of gemcitabine.

In a further aspect the invention provides use of a compound of formula I or a pharmaceutically acceptable derivative thereof in the preparation of a single-agent medicament for use in combination with a compound of formula II or pharmaceutically acceptable salt thereof or in the preparation of a combined medicament with the compound of formula II or pharmaceutically acceptable salt thereof, for the treatment of a neoplastic disease. In some embodiments the compound of formula I or pharmaceutically acceptable derivative thereof is a compound of formula I-A or a pharmaceutically acceptable derivative thereof. In further embodiments the compound of formula I or pharmaceutically acceptable derivative thereof is a compound of formula I-A or a pharmaceutically acceptable derivative thereof and the compound of formula II or pharmaceutically acceptable salt thereof is gemcitabine or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof and the compound of formula II or pharmaceutically acceptable salt thereof is gemcitabine or a pharmaceutically acceptable salt thereof. In further embodiments the compound of formula I or pharmaceutically acceptable derivative thereof is the dihydrochloride salt of the compound of formula I-B.

In further embodiments the compound of formula I or pharmaceutically acceptable derivative thereof is the dihydrochloride salt of the compound of formula I-B and the compound of formula II or pharmaceutically acceptable salt thereof is the hydrochloride salt of gemcitabine.

In a further aspect the invention provides use of a compound of formula II or a pharmaceutically acceptable salt thereof in the preparation of a single-agent medicament for use in combination with a compound of formula I or pharmaceutically acceptable derivative thereof or in the preparation of a combined medicament with the compound of formula I or pharmaceutically acceptable derivative thereof, for the treatment of a neoplastic disease. In some embodiments the compound of formula I or pharmaceutically acceptable derivative thereof is a compound of formula I-A or a pharmaceutically acceptable derivative thereof. In further embodiments the compound of formula I or pharmaceutically acceptable derivative thereof is a compound of formula I-A or a pharmaceutically acceptable derivative thereof and the compound of formula II or pharmaceutically acceptable salt thereof is gemcitabine or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof. In further embodiments the compound of formula I or pharmaceutically acceptable derivative thereof is a compound of formula I-A or a pharmaceutically acceptable salt thereof or a compound of formula I-B or a pharmaceutically acceptable salt thereof and the compound of formula II or pharmaceutically acceptable salt thereof is gemcitabine or a pharmaceutically acceptable salt thereof. In further embodiments the compound of formula I or pharmaceutically acceptable derivative thereof is the dihydrochloride salt of the compound of formula I-B.

In further embodiments the compound of formula I or pharmaceutically acceptable derivative thereof is the dihydrochloride salt of the compound of formula I-B and the compound of formula II or pharmaceutically acceptable salt thereof is the hydrochloride salt of gemcitabine.

Of particular interest is the compound of formula I-A (BAL27862) and pharmaceutically acceptable derivatives thereof. Examples of derivatives of the compound of formula I and in particular of the compound of formula I-A are described herein. Of particular interest is the compound of formula I-B (BAL101553, as indicated above) and pharmaceutically acceptable salts thereof. More particularly a dihydrochloride salt of the compound of formula I-B.

The compound of formula I or pharmaceutically acceptable derivative thereof and the compound of formula II or pharmaceutically acceptable salt thereof may be in the same pharmaceutical composition, e.g. as a single dosage unit, but will usually be provided in separate pharmaceutical compositions, e.g. as separate dosage units. Separate pharmaceutical compositions have a number of advantages, for example, to allow different dosing schedules, different dosages and/or different routes of administration for each compound. When provided as separate formulations the combination may be for separate, simultaneous or sequential administration.

Neoplastic diseases for treatment by combinations of the invention are described below, and are in particular contemplated for treatment of cancer, and in particular for human subjects.

Additional aspects and embodiments of the invention are described in more detail below.

### BRIEF DESCRIPTION OF FIGURES

FIG. 1, FIG. 2 and FIG. 3 show the results of an experiment comparing the efficacy and tolerability of the combination of BAL101553 with Gemcitabine versus the respective monotherapies in mice bearing the patient-derived pancreatic tumour PAXF1657 xenograft. BAL101553 was administered orally daily and Gemcitabine intravenously every week. In the combined regimen Gemcitabine was administered 4 hours before BAL101553 when administered on the same day. Control animals received vehicle control according to the applied single agent application schedules respectively. FIG. 1 shows the results of each of the single agent treatments. FIG. 2 shows the results of the combination treatment in which BAL101553 was administered at a dose of 8 mg/kg and Gemcitabine at a dose of 240 mg/kg. FIG. 3 shows the results of the combination treatment in which BAL101553 was administered at a dose of 10 mg/kg and Gemcitabine at 240 mg/kg. In each Figure the upper panel (FIGS. 1A, 2A and 3A) shows mean tumor volumes and the lower panel (FIGS. 1B, 2B and 2C) shows mean body weight changes. Data points represent mean values+/−SEM (n=10 animals).

FIG. 4 shows the determination of the pharmacokinetic profile of BAL27862, Gemcitabine and the combined treatment thereof in mouse plasma. PAXF1657 tumor bearing mice of the identical strain were treated either with BAL101553 oral 10 mg/kg, Gemcitabine 240 i.v. mg/kg or with a combination of BAL101553 and Gemcitabine where Gemcitabine was administered 4 hours before BAL101553. Control animals received appropriate vehicle control. The mice were dosed until steady state was reached (BAL101553 was dosed d1-d3 and Gemcitabine was dosed on day 3, the doses used for the combination-treatment matched those used in the monotherapy). Blood was taken from every animal according to a determined schedule covering a time window of 10 minutes to 24 hours, processed for plasma and subsequently analyzed by LC-MS/MS for the determination of BAL27862 (parent drug of BAL101553) and Gemcitabine concentrations (ng/ml plasma). Data points represent mean values of ng/ml plasma concentration+/−SEM (n=3 animals).

### DETAILED DESCRIPTION OF THE INVENTION

### Definitions

Certain terms used herein are described below. Compounds of the present invention are described using standard nomenclature. Unless defined otherwise, all technical and scientific terms used herein have the same meaning as is commonly understood by one of skill in the art to which this invention belongs.

The term derivative or derivatives in the phrase “pharmaceutically acceptable derivative” or “pharmaceutically acceptable derivatives” of compounds of formula I relates to pharmaceutically acceptable salts, pro-drugs and pharmaceutically acceptable salts of pro-drugs thereof.

The term “combination,” “therapeutic combination,” or “pharmaceutical combination” as used herein refer to either a fixed combination in one dosage unit form, or non-fixed combination, or a kit, e.g. a kit of parts, for the combined administration where two or more therapeutic agents may be administered independently, at the same time or separately within time intervals, especially where these time intervals allow that the combination partners show a cooperative, e.g. synergistic, effect. Usually components (a) and (b) will be provided as separate dosage forms for independent administration.

The term “combination therapy” refers to the administration of two or more therapeutic agents to treat a therapeutic condition or disorder described in the present disclosure. Such administration encompasses co-administration of these therapeutic agents in a substantially simultaneous manner as well as use of each type of therapeutic agent in a sequential and/or separate manner (e.g. according to different administration routes), either at approximately the same time or at different times, e.g. according to different dosage regimens. When the therapeutic agents are administered sequentially and/or separately the dosing schedules will be such that there is a therapeutic interaction between the therapeutic agents within the patient's body and/or that a therapeutic effect resulting from the first therapeutic agent is present when the second therapeutic agent is administered. For example, when the agents are administered according to cyclic treatment schedules, the cyclic treatment schedules may overlap, or when one therapeutic agent is administered according to a continuous dosing schedule and the second according to a cyclic schedule, then at least one dose from the agent administered according to the continuous schedule will occur during the treatment cycle of the other therapeutic agent. Usually there will be at least one interval of no more than seven days between doses of the two therapeutic agents.

The term “pharmaceutical composition” is defined herein to refer to a solid or liquid formulation containing at least one therapeutic agent to be administered to a subject, e.g. a mammal in particular a human, optionally with one or more pharmaceutically acceptable excipients, in order treat a particular disease or condition affecting the subject.

The term “pharmaceutically acceptable” as used herein refers to items such as compounds, materials, compositions and/or dosage forms, which are, within the scope of sound medical judgment, suitable for contact with the tissues of a warm-blooded animal, e.g. a mammal in particular a human, without excessive toxicity or other complications commensurate with a reasonable benefit/risk ratio.

The terms “fixed combination,” “fixed dose,” and “single formulation” as used herein refers to a single carrier or vehicle or dosage form formulated to deliver an amount, which is jointly therapeutically effective for the treatment of neoplastic diseases, of both therapeutic agents to a patient. The single vehicle is designed to deliver an amount of each of the agents, along with any pharmaceutically acceptable carriers or excipients.

The term “non-fixed combination,”, “kit”, and “separate formulations” means that the active ingredients, i.e., the compound of formula I or pharmaceutically acceptable derivative and compound of formula II (gemcitabine, e.g. gemcitabine hydrochloride), are both administered to a patient as separate entities either simultaneously, concurrently or sequentially, wherein such administration provides therapeutically effective levels of the two compounds in the body of the subject in need thereof.

The term “treatment,” as used herein in the context of treating a neoplastic disease in a patient pertains generally to treatment and therapy in which some desired therapeutic effect is achieved, for example one or more of the following: the inhibition of the progress of the neoplastic disease, a reduction in the rate of progress, a halt in the rate of progress, a prevention of the progression of the neoplastic disease, alleviation of symptoms of the neoplastic disease, amelioration of neoplastic disease, and cure of the neoplastic disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

The term “prevent”, “preventing” or “prevention” as used herein comprises the prevention of at least one symptom associated with or caused by the state, disease or disorder being prevented.

The term “pharmaceutically effective amount,” “therapeutically effective amount,” or “clinically effective amount” of a combination of therapeutic agents is an amount sufficient to provide an observable or clinically significant improvement over the baseline clinically observable signs and symptoms of the disorders treated with the combination.

The term “subject” or “patient” as used herein is intended to include animals, which are capable of suffering from or afflicted with a neoplastic disease such as a cancer or any disorder involving, directly or indirectly, a neoplastic disease such as a cancer. Examples of subjects include mammals, e.g. humans, apes, monkeys, dogs, cows, horses, pigs, sheep, goats, cats, mice, rabbits, rats, and transgenic non-human animals. Preferably, the subject is a human, e.g. a human suffering from, at risk of suffering from neoplastic diseases such as cancers.

The term “therapeutically-effective amount,” as used herein, pertains to that amount of a therapeutic agent, or a material, composition or dosage form comprising a therapeutic agent, which is effective for producing some desired therapeutic effect, commensurate with a reasonable benefit/risk ratio, when administered in accordance with a desired treatment regimen. The skilled person will understand that the therapeutically effective amount of an agent for use in combination therapy may be lower than the amount required to provide a therapeutic effect when using the agent as a monotherapy.

The term “about” means a variation of no more than 10% of the relevant figure, preferably no more than 5%.

For convenience, reference to the compound of formula I refers to the compound e.g. in free form and pharmaceutically acceptable salts thereof. Reference to derivatives of the compound of formula I refers to the derivatives e.g. in free form and pharmaceutically acceptable salts of said derivatives. Likewise, reference to the compound of formula II (gemcitabine) refers to the compound e.g. in free form and pharmaceutically acceptable salts thereof and in particular the hydrochloride salt.

All possible solvates and complexes (including hydrates) of the compound of formula I and derivatives thereof as well as any polymorphs of the compound of formula I and derivatives thereof, including amorphous solids, as well as pharmaceutically acceptable salts of any of the foregoing are included within the scope of the invention. Likewise all possible solvates and complexes (including hydrates) of the compound of formula II as well as any polymorphs of the compound of formula I and derivatives thereof, including amorphous solids, as well as pharmaceutically acceptable salts of any of the foregoing are included within the scope of the invention.

Compound of Formula I and Derivatives Thereof

In some embodiments R is phenyl or phenyl substituted by one or two substituents independently selected from, methyl, ethyl, propyl, iso-propyl, methoxy, ethoxy, hydroxyl, amino, methylamino, ethylamino, dimethylamino, diethylamino, acetylamino, halogen (e.g. F, Cl or Br) and nitro.

In some embodiments R is pyridinyl or pyridinyl substituted by a single substituent selected from amino, F, Cl or Br.

In some embodiments R is phenyl or pyridinyl substituted by amino.

In some embodiments R¹ is H or cyanoethyl.

Preferred compounds of formula I include those wherein R and R¹ are defined as follows:

| R | R¹ |
|---|---|
| 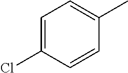 | H |
| 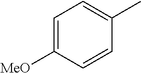 | H |
| 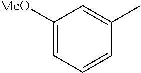 | H |
| 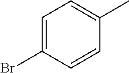 | H |
| 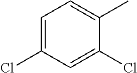 | H |
| 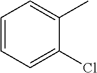 | H |
| 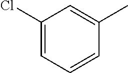 | H |
| 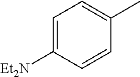 | H |
| 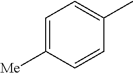 | H |
| 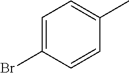 | CH₂CH₂CN |
| 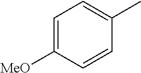 | CH₂CH₂CN |
| 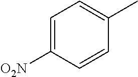 | H |
| 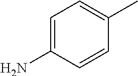 | H |
| 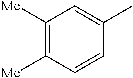 | H |
| 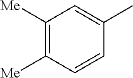 | CH₂CH₂CN |
| 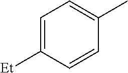 | H |
| 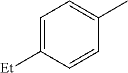 | CH₂CH₂CN |
| 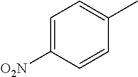 | CH₂CH₂CN |
| 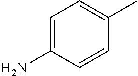 | CH₂CH₂CN |
| 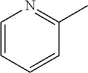 | H |
| 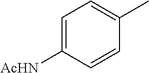 | H |
| 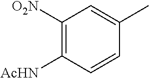 | H |
| 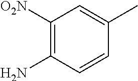 | H |
| 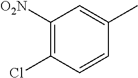 | H |
| 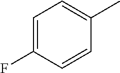 | H |
| 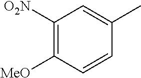 | H |
| 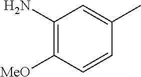 | CH₂CH₂CN |
| 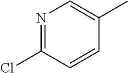 | H |
| 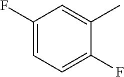 | H |
| 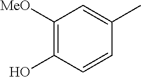 | H |
| 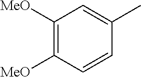 | H |
| 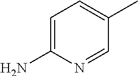 | H |
| 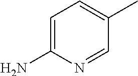 | CH₂CH₂CN |
| 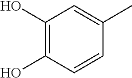 | H |

or pharmaceutically acceptable derivative thereof.

Especially preferred are compounds wherein R and R¹ are defined as follows:

| R | R¹ |
|---|---|
| 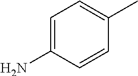 | H |
| 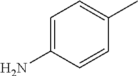 | CH₂CH₂N |
| 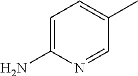 | H |
| 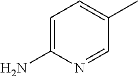 | CH₂CH₂CN |

or pharmaceutically acceptable derivative thereof.

An especially preferred compound is the compound of formula I-A or pharmaceutically acceptable derivative thereof:

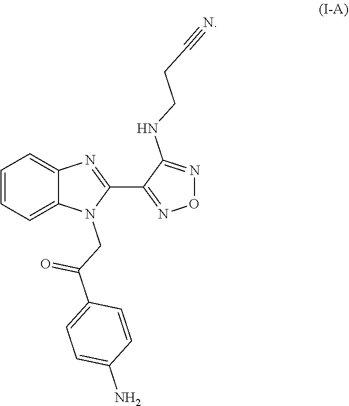 [SMILES: N#CCCNc1nonc1-c1nc2ccccc2n1CC(=O)c1ccc(N)cc1]

In some embodiments the compound of the formula I is the compound of formula I-A, or a pharmaceutically acceptable salt thereof.

In some embodiments the compound of the formula I is a prodrug of the compound of formula I-A, or a pharmaceutically acceptable salt of the prodrug.

Salts of the compound of formula I may be acid addition salts. Salts are formed, e.g. with organic or inorganic acids, from compounds of formula I or pharmaceutically acceptable derivatives thereof with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

The compound of formula I may be administered in the form of a pharmaceutically acceptable derivative. Suitably the compound of formula I, in particular the compound of formula I-A, is administered in the form of a pro-drug, including pharmaceutically acceptable salts thereof, which is broken down in the subject (e.g. human) to give a compound of the formula I. Examples of pro-drugs include in vivo hydrolysable esters and amides of a compound of the formula I. Particular pro-drugs considered are ester and amides of naturally occurring amino acids and ester or amides of small peptides, in particular small peptides consisting of up to five, preferably two or three amino acids as well as esters and amides of pegylated hydroxy acids, preferably hydroxy acetic acid and lactic acid. Pro-drug esters may be formed from the acid function of the amino acid or the C terminal of the peptide and suitable hydroxy group(s) in the compound of formula I. Pro-drug amides may be formed from the acid function of the amino acid or the C terminal of the peptide and suitable amino group(s) in the compound of formula I. In particular, the pro-drug amides are formed from the amino group(s) present within the R group of formula I, e.g. the pro-drug is an amide formed from an amino group present within the R group of the compound of formula I as defined above and the carboxy group of glycine, alanine or lysine.

The compound of formula I may be in the form of a pro-drug selected from the compounds of the following formulae and pharmaceutically acceptable salts thereof:

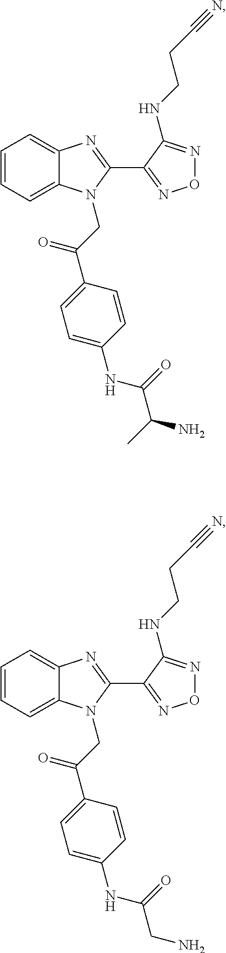 [SMILES: C[C@H](N)C(=O)Nc1ccc(C(=O)Cn2c(-c3nonc3NCCC#N)nc3ccccc32)cc1 ; N#CCCNc1nonc1-c1nc2ccccc2n1CC(=O)c1ccc(NC(=O)CN)cc1]

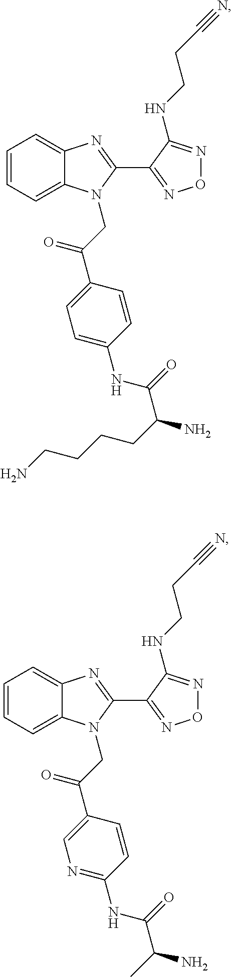 [SMILES: N#CCCNc1nonc1-c1nc2ccccc2n1CC(=O)c1ccc(NC(=O)[C@@H](N)CCCCN)cc1 ; C[C@H](N)C(=O)Nc1ccc(C(=O)Cn2c(-c3nonc3NCCC#N)nc3ccccc32)cn1]

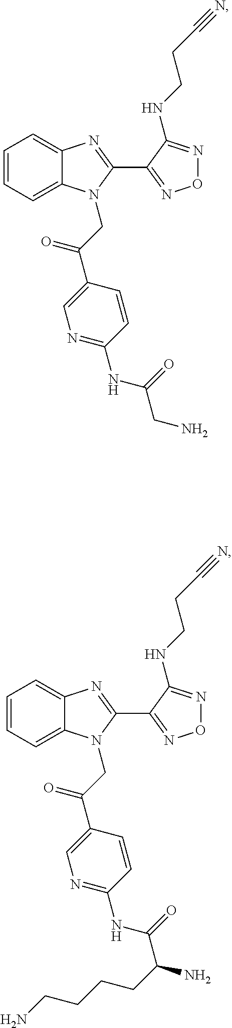 [SMILES: N#CCCNc1nonc1-c1nc2ccccc2n1CC(=O)c1ccc(NC(=O)CN)nc1 ; N#CCCNc1nonc1-c1nc2ccccc2n1CC(=O)c1ccc(NC(=O)[C@@H](N)CCCCN)nc1]

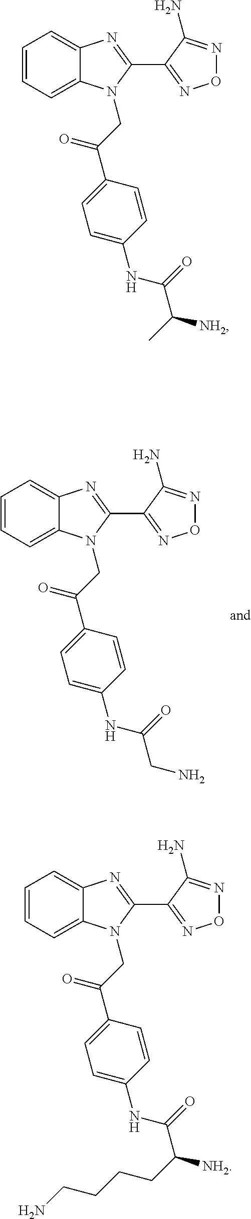 [SMILES: C[C@H](N)C(=O)Nc1ccc(C(=O)Cn2c(-c3nonc3N)nc3ccccc32)cc1 ; NCC(=O)Nc1ccc(C(=O)Cn2c(-c3nonc3N)nc3ccccc32)cc1 ; NCCCC[C@H](N)C(=O)Nc1ccc(C(=O)Cn2c(-c3nonc3N)nc3ccccc32)cc1]

When the compound of formula I is provided as a pro-drug it is preferably the compound of formula I-B or a pharmaceutically acceptable salt thereof, e.g. a hydrochloride salt such as a dihydrochloride salt.

Reference to a compound of formula I or pharmaceutically acceptable derivative thereof preferably refers to a compound of formula I-A or pharmaceutically acceptable salt thereof, or a compound of formula I-B or pharmaceutically acceptable salt thereof.

The compounds of formula I may be prepared as described in WO2004/103994, which is hereby incorporated by reference. The derivatives of the compound of formula I, in particular the pro-drugs of the compound of formula I, may be prepared as described for example in WO2011/012577, in particular on pages 29 to 39, which is hereby incorporated by reference.

Compound of Formula II (Gemcitabine)

The terms “compound of formula II” and “gemcitabine” are used herein interchangeably. Methods for the synthesis of gemcitabine are described, for example, in EP0184365, Brown et al., “A linear synthesis of gemcitabine”, Carbohydrate Research, vol. 406, pages 71-75, 2015, Brown et al., “The synthesis of gemcitabine”, Carbohydrate Research, vol. 387, pages 59-73, 2014, which are incorporated herein by reference. Gemcitabine is available commercially as the hydrochloride salt and is marketed e.g. as Gemzar®. Gemcitabine has the CAS Registry number 95058-81-4 and the hydrochloride salt has the CAS Registry number CAS122111-03-9.

Diseases

The pharmaceutical combinations of the invention may be used to treat neoplastic diseases by administration of the combinations of the invention, e.g. to destabilize the microtubules (compound of formula I) and inhibit DNA synthesis (compound of formula II). In addition, the pharmaceutical combinations of the invention may be used to treat a cancer at any clinical stage or pathological grade (e.g. tumour stage I, tumour stage II, tumour stage III, tumour stage IV) or treatment settings (e.g. preventative, adjuvant, neoadjuvant, therapeutic including palliative treatment). The pharmaceutical combinations of the invention may be for use in slowing, delaying or stopping cancer progression or cancer growth or increasing the overall survival time or the cancer-progression-free survival time or the time to progression of a cancer or improving or maintaining the patient's quality of life or functional status. The pharmaceutical combinations of the invention may also be used in post-therapy recovery from cancer.

For example, the pharmaceutical combinations of the invention of the invention may be used for (i) reducing the number of cancer cells; (ii) reducing tumour volume; (iii) increasing tumour regression rate; (iv) reducing or slowing cancer cell infiltration into peripheral organs; (v) reducing or slowing tumour metastasis; (vi) reducing or inhibiting tumour growth; (vii) preventing or delaying occurrence and/or recurrence of the cancer and/or extends disease- or tumour-free survival time; (viii) increasing overall survival time; (ix) reducing the frequency of treatment; and/or (x) relieving one or more of symptoms associated with the cancer.

As mentioned above, the pharmaceutical combinations of the invention may be used for the therapeutic treatment of neoplastic diseases. Examples of neoplastic diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous neoplasms, meningiomas, nerve sheath tumours, granular cell tumours and alveolar soft part sarcomas, Hodgkin's and non-Hodgkin's lymphomas, other lymphoreticular neoplasms, plasma cell tumours, mast cell tumours, immunoproliferative diseases, leukemias, miscellaneous myeloproliferative disorders, lymphoproliferative disorders and myelodysplastic syndromes.

In one embodiment the neoplastic disease is cancer. Examples of cancers in terms of the organs and parts of the body affected include, but are not limited to, the brain, breast (including triple negative breast cancer), cervix, ovaries, colon, rectum, (including colon and rectum i.e. colorectal cancer), lung, (including small cell lung cancer, non-small cell lung cancer, large cell lung cancer and mesothelioma), endocrine system, bone, adrenal gland, thymus, liver, stomach, intestine, (including gastric cancer), pancreas, bone marrow, haematological malignancies, (such as lymphoma, leukaemia, myeloma or lymphoid malignancies), bile duct, bladder, urinary tract, kidneys, skin, thyroid, head, neck, prostate and testis.

For example, the cancer may be selected from the group consisting of brain cancer (e.g. neuroblastoma, glioblastoma), breast cancer (including triple negative breast cancer), prostate cancer, cervical cancer, ovarian cancer (including ovarian carcinoma), biliary cancer, gastric cancer, colorectal cancer, pancreatic cancer, liver cancer, neuroendocrine cancer, lung cancer, kidney cancer, bladder cancer, haematological malignancies, melanoma and sarcomas (including soft tissue sarcomas, e.g. liposarcoma).

The cancer may be for example a primary tumour, or metastases, derived for example from a solid or liquid tumour. In one embodiment the neoplastic disease (e.g. cancer) to be treated is a tumour, preferably a solid tumour.

In a further embodiment the neoplastic disease is a brain neoplasm, e.g. a brain tumour, which include but are not limited to glial- and non-glial-tumours, astrocytomas (incl. glioblastoma multiforme and unspecified gliomas), oligodendrogliomas, ependydomas, menigiomas, haemangioblastomas, acoustic neuromas, craniopharyngiomas, primary central nervous system lymphoma, germ cell tumours, pituitary tumours, pineal region tumours, primitive neuroectodermal tumours (PNET's), medullablastomas, haemangiopericytomas, spinal cord tumours including meningiomas, chordomas and genetically-driven brain neoplasms including neurofibromatosis, peripheral nerve sheath tumours and tuberous sclerosis.

In a further embodiment the cancer is brain cancer (e.g. neuroblastoma, glioblastoma).

In a further embodiment the cancer is breast cancer (including triple negative breast cancer, hormone receptor positive breast cancer and HER2 positive breast cancer).

In a further embodiment the cancer is prostate cancer.

In a further embodiment the cancer is cervical cancer.

In a further embodiment the cancer is ovarian cancer (including ovarian carcinoma).

In a further embodiment the cancer is gastric cancer.

In a further embodiment the cancer is colorectal cancer.

In a further embodiment the cancer is pancreatic cancer (including ductal adenocarcinoma and metastatic pancreatic cancer).

In a further embodiment the cancer is liver cancer.

In a further embodiment the cancer is neuroendocrine cancer.

In a further embodiment the cancer is lung cancer.

In a further embodiment the cancer is kidney cancer.

In a further embodiment the cancer is haematological malignancies.

In a further embodiment the cancer is melanoma.

In a further embodiment the cancer is a sarcoma.

In a further embodiment the cancer is non-small cell lung cancer.

In a further embodiment the cancer is mesothelioma.

In a further embodiment the cancer is bladder cancer.

In a further embodiment the cancer is biliary tract cancer.

In a further embodiment the cancer is peripheral or cutaneous T-cell lymphoma.

In a further embodiment the cancer is non-Hodgkins lymphoma.

In a further embodiment the cancer is a cancer selected from lung cancer (including non-small cell lung cancer and mesothelioma), pancreatic cancer (including ductal adenocarcinoma and metastatic pancreatic cancer), bladder cancer, ovarian cancer (including ovarian carcinoma), biliary tract cancer, peripheral or cutaneous T-cell lymphoma, non-Hodgkins lymphoma and breast cancer.

In a further embodiment the cancer is a pancreatic cancer, in particular metastatic pancreatic cancer.

Administration

Administration of the pharmaceutical combinations of the invention includes administration of the combination in a single formulation or unit dosage form, as well as administration of the individual agents of the combination in separate formulations or separate dosage forms.

The present invention particularly pertains to a combination of the invention for treating cancer. In an embodiment, the combination of the invention is used for the treatment of cancer comprising administering to the subject a combination therapy, comprising a therapeutically effective amount of a compound of formula I (e.g. the compound of formula I-A or pharmaceutically acceptable salt thereof) or pharmaceutically acceptable derivative thereof (e.g. the compound of formula I-B or pharmaceutically acceptable salt thereof), and a therapeutically effective amount of the compound of formula II or a pharmaceutically acceptable salt thereof (e.g. gemcitabine hydrochloride). These compounds are administered at therapeutically effective dosages, which when combined may provide a beneficial effect e.g. as described herein. The skilled person will understand that therapeutically effective dosages for use in combination therapy may be lower than the dosages required to provide a therapeutic effect when using either agent as a monotherapy.

The administration of a pharmaceutical combination of the invention may result not only in a beneficial effect, e.g. a synergistic effect, e.g. with regard to alleviating, delaying progression of or inhibiting the symptoms, but may also result in further beneficial effects, e.g. fewer side-effects, more durable therapeutic effect, an improved quality of life or a decreased morbidity, compared with a monotherapy applying only one of the pharmaceutically therapeutic agents used in the combination of the invention. It may also be the case that lower doses of the therapeutic agents of the combination of the invention can be used, for example, such that the dosages may not only often be smaller, but also may be applied less frequently, or can be used in order to diminish the incidence of side-effects observed with one of the combination partners alone.

In an embodiment, the combination provided herein may display a synergistic effect. The term “synergistic effect” as used herein, refers to action of two agents such as, for example, the compound of formula I (e.g. the compound of formula I-A or pharmaceutically acceptable salt thereof) or pharmaceutically acceptable derivative thereof (e.g. the compound of formula I-B or pharmaceutically acceptable salt thereof) and a compound of formula II or pharmaceutically acceptable salt thereof (e.g. gemcitabine hydrochloride), to produce a therapeutic effect, e.g. slowing the progression of a neoplastic disease such as cancer or symptoms thereof, which is greater than the addition of the same therapeutic effect of each drug administered on its own.

Generally, in determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in certain experiments can be predictive of the effect in other species, and animal models may be used to further quantify a synergistic effect. The results of such studies can also be used to predict effective dose ratio ranges and the absolute doses and plasma concentrations, e.g. as illustrated in the Examples below.

In a further embodiment, the present invention provides a synergistic combination for administration to humans comprising the pharmaceutical combination of the invention, where the dose range of each component corresponds to the synergistic ranges, e.g. as indicated in a suitable tumour model or clinical study.

The combinations of the present invention can be used in long-term therapy or as an adjuvant therapy in the context of other treatment strategies, as described above. Other possible treatments are therapy to maintain the patient's status after tumour regression, or even preventive therapy, for example in patients at risk.

The compound of formula I or pharmaceutically acceptable derivative thereof and the compound of formula II or pharmaceutically acceptable salt thereof may be administered according to the same treatment schedule or may be administered according to independent treatment schedules. The treatment schedules may be cyclic or continuous.

A cyclic treatment schedule is defined by a repeated dosing schedule wherein the repeated element (a cycle) has a specific duration and wherein doses are administered on specific days within the cycle. A cycle may incorporate a period, usually at the end of the cycle, in which there is no administration (a “rest period”), e.g. to allow a period for recovery. A treatment cycle may be, e.g. 7 days, 14 days, 21 days, 28 days or longer. For example, the Federal Drug Administration and European Medicines Agency have recommended that gemcitabine is administered on days 1 and 8 of a 21-day treatment cycle. Accordingly, in this example of a cyclic treatment schedule gemcitabine is administered once per week for two weeks followed by a rest period of 1 week (i.e. no gemcitabine is administered on days 9 to 21 of the cycle). If the cycle is repeated further gemcitabine would be administered according to the cycle on the day following day 21 of the cycle.

A continuous treatment schedule is a regular dosing schedule which does not incorporate rest periods (i.e. periods that are longer than the regular interval between the doses). For example doses may be administered once per day, twice per day, once every two days, once every three days etc.

The treatment schedule, whether cyclic or continuous may be continued for as long as required (an “open-end treatment”) e.g. as long as the patient is receiving benefit judged by a physician overseeing the treatment.

When the compound of formula I or pharmaceutically acceptable derivative thereof and the compound of formula II or pharmaceutically acceptable salt thereof are administered according to independent treatment schedules, the treatment schedules may both be cyclic, or one may be cyclic and the other may be continuous. When both treatment schedules are cyclic, the cycles of the two treatment schedules may be of the same duration or may be of different duration, and they may start on the same day or may start on different days.

In another embodiment the compound of formula I or pharmaceutically acceptable derivative thereof is administered according to a continuous treatment schedule, and the compound of formula II or pharmaceutically acceptable salt thereof is administered according to a cyclic treatment schedule wherein each cycle has a duration of 21 days. The treatment schedules may start on the same day or may start on different days.

In another embodiment the compound of formula I or pharmaceutically acceptable derivative thereof is administered according to a continuous treatment schedule, and the compound of formula II or pharmaceutically acceptable salt thereof is administered according to a cyclic treatment schedule wherein each cycle has a duration of 28 days. The treatment schedules may start on the same day or may start on different days.

In another embodiment the compound of formula I or pharmaceutically acceptable derivative thereof and the compound of formula II or pharmaceutically acceptable salt thereof are both administered according to a cyclic treatment schedule wherein each cycle has a duration of 21 days, and which treatment schedules may start on the same day or may start on different days.

In another embodiment the compound of formula I or pharmaceutically acceptable derivative thereof is administered according to a cyclic treatment wherein each cycle has a duration of 28 days, and the compound of formula II or pharmaceutically acceptable salt thereof is administered according to a cyclic treatment schedule wherein each cycle has a duration of 21 days. The treatment schedules may start on the same day or may start on different days.

In another embodiment the compound of formula I or pharmaceutically acceptable derivative thereof is administered according to a cyclic treatment wherein each cycle has a duration of 21 days, and the compound of formula II or pharmaceutically acceptable salt thereof is administered according to a cyclic treatment schedule wherein each cycle has a duration of 28 days. The treatment schedules may start on the same day or may start on different days.

In another embodiment the compound of formula I or pharmaceutically acceptable derivative thereof and the compound of formula II or pharmaceutically acceptable salt thereof are both administered according to a cyclic treatment schedule wherein each cycle has a duration of 28 days, and which treatment schedules may start on the same day or may start on different days.

Additional embodiments are provided in Table A below.

TABLE A

| | Compound of formula IA | |
|---|---|---|
| | or pharmaceutically acceptable | |
| | salt thereof or compound of | Compound of formula II |
| | formula IB or pharmaceutically | or pharmaceutically |
| Embodiment | acceptable salt thereof | acceptable salt thereof |
| 1A | Oral administration: continuous treatment | IV administration: cyclic treatment schedule, 21- |
| | schedule, dosing every day | day treatment cycle, dosing initiated on days 1 and |
| | | 8 |
| 2A | Oral administration: continuous treatment | IV administration: cyclic treatment schedule, 28- |
| | schedule, dosing every day | day treatment cycle, dosing initiated on days 1, 8 and |
| | | 15 |
| 3A | Oral administration: continuous treatment | IV administration: cyclic treatment schedule, 28- |
| | schedule, dosing every day | day treatment cycle, dosing initiated on days 1, 8, 15 |
| | | and 21 |
| 4A* | IV administration: cyclic treatment schedule, | IV administration: cyclic treatment schedule, 21- |
| | 21-day treatment cycle, dosing initiated on | day treatment cycle, dosing initiated on days 1 and |
| | days 1 and 8 | 8 |
| 5A* | IV administration: cyclic treatment schedule, | IV administration: cyclic treatment schedule, 21- |
| | 28-day treatment cycle, dosing initiated on | day treatment cycle, dosing initiated on days 1 and |
| | days 1, 8 and 15 | 8 |
| 6A* | IV administration: cyclic treatment schedule, | IV administration: cyclic treatment schedule, 28- |
| | 21-day treatment cycle, dosing initiated on | day treatment cycle, dosing initiated on days 1, 8 |
| | days 1 and 8 | and 15 |
| 7A* | IV administration: cyclic treatment schedule, | IV administration: cyclic treatment schedule, 28- |
| | 28-day treatment cycle, dosing initiated on | day treatment cycle, dosing initiated on days 1, 8 |
| | days 1, 8 and 15 | and 15 |
| 8A* | IV administration: cyclic treatment schedule, | IV administration: cyclic treatment schedule, 28- |
| | 21-day treatment cycle, dosing initiated on | day treatment cycle, dosing initiated on days 1,, 8, 15 |
| | days 1 and 8 | and 21 |
| 9A* | IV administration: cyclic treatment schedule, | IV administration: cyclic treatment schedule, 28- |
| | 28-day treatment cycle, dosing initiated on | day treatment cycle, dosing initiated on days 1,, 8, 15 |
| | days 1, 8 and 15 | and 21 |

*Where cyclic treatment schedules are used for both combination partners, cycles may or may not start on the same day

Examples of mole dosage ratios (i.e. number of moles: number of moles) of the compound of formula II or pharmaceutically acceptable salt thereof to the compound of formula I or pharmaceutically acceptable derivative thereof e.g. 2:1 to 340:1, e.g. 5:1 to 95:1, e.g. 5:1 to 85:1, e.g. 5:1 to 65:1, e.g. 5:1 to 45:1, e.g. 10:1 to 85:1. For example the mole:mole ratio of compound of formula II or pharmaceutically acceptable salt thereof to the compound of formula I or pharmaceutically acceptable derivative thereof may be e.g. at least 2:1, e.g. at least 5:1, e.g. at least 10:1. For example the mole:mole ratio of the compound of formula II or pharmaceutically acceptable salt thereof to the compound of formula I or pharmaceutically acceptable derivative thereof may be e.g. up to 340:1, e.g. up to 95:1, e.g. up to 65:1, e.g. up to 85:1, e.g. up to 45:1.

If a cyclic treatment schedule is used for both combination partners and the cycles are of the same duration then the mole ratio is the ratio of the total mole amount of the doses of the compound of formula II or pharmaceutically acceptable salt thereof to the total mole amount of the doses of the compound of formula I or pharmaceutically acceptable derivative thereof administered to the subject over the respective cycles.

If a cyclic treatment schedule is used for both combination partners and the cycles are of different duration then the mole ratio is the ratio of the total mole amount of the doses of the compound of formula II or pharmaceutically acceptable salt thereof to the total mole amount of the doses of the compound of formula I or pharmaceutically acceptable derivative thereof administered to the subject over a theoretical period of time corresponding to a common multiple of the duration of the respective treatment cycles. For example if one combination partner is administered according to a three-week cycle and the other combination partner is administered according to a four-week cycle, then the total number of administrations over a 12-week period (i.e. three cycles of the four week cycle and four cycles of the three week cycle) will be used to determine the mole ratio.

If a continuous treatment schedule is used for one combination partner and a cyclic treatment schedule is used for the other combination partner then the mole ratio is the ratio of the total mole amount of the doses of the combination partner administered to the subject according to a continuous treatment over a period of the same duration of one cycle of the other combination partner to the total mole amount of the doses of the other combination partner administered to the subject over the treatment cycle. For example if the treatment cycle of one of the combination partners has a duration of 21 days, then for the determination of the mole ratio the total mole amount of the doses of the other combination partner over a period of 21 days is used, e.g. if administered once every two days, then the sum of the mole amount of 11 doses will be used for the determination of the mole ratio. In the event that the arrangement of the dosing schedules leads to the possibility of different numbers of doses of the continuous treatment over the duration of the cycle of the cyclic treatment, then the larger number of doses of the continuous treatment is used for the determination of the mole ratio.

If a continuous treatment schedule is used for both combination partners, then the respective total mole amount of the doses over a period of seven days is used to determine the mole ratios. For example if one combination partner is administered once every two days and the other is administered once every seven days, then for the determination of the above ratios, the sum of the mole amount of four doses of the first combination partner versus one dose of the second combination partner is used to determine the mole ratio.

Examples of weight:weight dosage ratios of the compound of formula II (in the form of gemcitabine hydrochloride) to the compound of formula I-B (in the form of the dihydrochloride salt) may be e.g. 3:1 to 670:1, e.g. 10:1 to 190:1, e.g. 10:1 to 170:1, e.g. 10:1 to 130:1, e.g. 10:1 to 90:1, e.g. 20:1 to 170:1. For example, the weight:weight dosage ratio of the compound of formula II (in the form of gemcitabine hydrochloride) to the compound of formula I-B (in the form of the dihydrochloride salt) may be e.g. at least 3:1, e.g. at least 10:1, e.g. at least 20:1. For example, the weight:weight dosage ratio of the compound of formula II (in the form of gemcitabine hydrochloride) to the compound of formula I-B (in the form of the dihydrochloride salt) may be e.g. up to 670:1, e.g. up to 190:1, e.g. up to 170:1, e.g. up to 130:1, e.g. up to 90:1. When the compound of formula I-B is provided in a form other than the dihydrochloride salt, e.g. as a different pharmaceutically acceptable salt, and/or when gemcitabine is provided in a form other than the hydrochloride salt, e.g. as a different pharmaceutically acceptable salt, then the corresponding weight:weight ratios giving the same mole:mole ratio apply, based on the respective molecular weights.

The molecular weight of the dihydrochloride salt of the compound of formula I-B is 588.5 Da and the molecular weight of gemcitabine hydrochloride is 299.7 Da.

The weight:weight dosage ratios are determined according to the same methodology as given for mole dosage ratios described above, i.e. the ratio of the total weight amount of the doses of the compound of formula II or pharmaceutically acceptable salt thereof to the total weight amount of the doses of the compound of formula I or pharmaceutically acceptable derivative thereof over the respective treatment cycles, when cyclic treatment schedules are used for both combination partners. Likewise, if a continuous treatment schedule is used for one combination partner and a cyclic treatment schedule is used for the other combination partner then the total weight amount of the doses of the continuous treatment over a period of the same duration of the treatment cycle of the other combination partner is used to determine the weight:weight ratio.

Examples of weight:weight dosage ratios of the compound of formula II (in the form of gemcitabine hydrochloride) to the compound of the compound of formula I-A (in free form) may be e.g. 2:1 to 440:1, e.g. 5:1 to 120:1, e.g. 5:1 to 110:1, e.g. 5:1 to 85:1, e.g. 5:1 to 60:1, e.g 10:1 to 110:1. For example the weight:weight dosage ratios of the compound of formula II (in the form of gemcitabine hydrochloride) to the compound of the compound of formula I-A (in free form) may be e.g. at least 2:1 e.g. at least 5:1, e.g. at least 10:1. For example the weight:weight dosage ratios of the compound of formula II (in the form of gemcitabine hydrochloride) to the compound of the compound of formula I-A (in free form) may be e.g. up to 440:1, e.g. up to 120:1, e.g. up to 110:1, e.g. up to 85:1, up to 60:1. Likewise, when the compound of formula I-A is provided in a form other than the free form, e.g. as a pharmaceutically acceptable salt or as a prodrug or salt thereof, and/or when gemcitabine is provided in a form other than the hydrochloride salt, e.g. as a different pharmaceutically acceptable salt, then corresponding weight:weight ratios giving the same mole:mole ratio apply based on the respective molecular weights. The molecular weight of the free form of the compound of formula I-A is 387.4 Da and the molecular weight of gemcitabine hydrochloride is 299.7 Da. Similarly, the weight:weight dosage ratios are determined according to the same methodology as given for mole ratios above.

Additional embodiments of the invention are shown in Table B and Table C below.

TABLE B

| | Combination | Combination | Mole ratio |
|---|---|---|---|
| Embodiment | partner I (CPI) | partner II (CPII) | of CPII:CPI |
| 1B | I-A or pharmaceutically | Gemcitabine or | 2:1 to 340:1 |
| | acceptable salt thereof or I-B or | pharmaceutically acceptable salt | |
| | pharmaceutically acceptable salt | thereof | |
| | thereof | | |
| 2B | I-A or pharmaceutically | Gemcitabine or | 5:1 to 95:1 |
| | acceptable salt thereof or I-B or | pharmaceutically acceptable salt | |
| | pharmaceutically acceptable salt | thereof | |
| | thereof | | |
| 3B | I-A or pharmaceutically | Gemcitabine or | 5:1 to 85:1 |
| | acceptable salt thereof or I-B or | pharmaceutically acceptable salt | |
| | pharmaceutically acceptable salt | thereof | |
| | thereof | | |
| 4B | I-A or pharmaceutically | Gemcitabine or | 5:1 to 65:1 |
| | acceptable salt thereof or I-B or | pharmaceutically acceptable salt | |
| | pharmaceutically acceptable salt | thereof | |
| | thereof | | |
| 5B | I-A or pharmaceutically | Gemcitabine or | 5:1 to 45:1 |
| | acceptable salt thereof or I-B or | pharmaceutically acceptable salt | |
| | pharmaceutically acceptable salt | thereof | |
| | thereof | | |
| 6B | I-A or pharmaceutically | Gemcitabine or | 10:1 to 85:1 |
| | acceptable salt thereof or I-B or | pharmaceutically acceptable salt | |
| | pharmaceutically acceptable salt | thereof | |
| | thereof | | |
| 7B | I-B or pharmaceutically | Gemcitabine or | 2:1 to 340:1 |
| | acceptable salt thereof | pharmaceutically acceptable salt | |
| | | thereof | |
| 8B | I-B or pharmaceutically | Gemcitabine or | 5:1 to 95:1 |
| | acceptable salt thereof | pharmaceutically acceptable salt | |
| | | thereof | |
| 9B | I-B or pharmaceutically | Gemcitabine or | 5:1 to 85:1 |
| | acceptable salt thereof | pharmaceutically acceptable salt | |
| | | thereof | |
| 10B | I-B or pharmaceutically | Gemcitabine or | 5:1 to 65:1 |
| | acceptable salt thereof | pharmaceutically acceptable salt | |
| | | thereof | |
| 11B | I-B or pharmaceutically | Gemcitabine or | 5:1 to 45:1 |
| | acceptable salt thereof | pharmaceutically acceptable salt | |
| | | thereof | |
| 12B | I-B or pharmaceutically | Gemcitabine or | 10:1 to 85:1 |
| | acceptable salt thereof | pharmaceutically acceptable salt | |
| | | thereof | |
| 13B | I-B as dihydrochloride salt | Gemcitabine hydrochloride | 2:1 to 340:1 |
| 14B | I-B as dihydrochloride salt | Gemcitabine hydrochloride | 5:1 to 95:1 |
| 15B | I-B as dihydrochloride salt | Gemcitabine hydrochloride | 5:1 to 85:1 |
| 16B | I-B as dihydrochloride salt | Gemcitabine hydrochloride | 5:1 to 65:1 |
| 17B | I-B as dihydrochloride salt | Gemcitabine hydrochloride | 5:1 to 45:1 |
| 18B | I-B as dihydrochloride salt | Gemcitabine hydrochloride | 10:1 to 85:1 |

TABLE C

| | | | Weight:weight |
|---|---|---|---|
| | Combination | Combination | ratio of |
| Embodiment | partner I (CPI) | partner II (CPII) | CPII:CPI |
| 1C* | I-A or pharmaceutically | Gemcitabine or | 3:1 to 670:1 |
| | acceptable salt thereof or I-B or | pharmaceutically acceptable salt | |
| | pharmaceutically acceptable salt | thereof | |
| | thereof | | |
| 2C* | I-A or pharmaceutically | Gemcitabine or | 10:1 to 190:1 |
| | acceptable salt thereof or I-B or | pharmaceutically acceptable salt | |
| | pharmaceutically acceptable salt | thereof | |
| | thereof | | |
| 3C* | I-A or pharmaceutically | Gemcitabine or | 101: to 170:1 |
| | acceptable salt thereof or I-B or | pharmaceutically acceptable salt | |
| | pharmaceutically acceptable salt | thereof | |
| | thereof | | |
| 4C* | I-A or pharmaceutically | Gemcitabine or | 10:1 to 130:1 |
| | acceptable salt thereof or I-B or | pharmaceutically acceptable salt | |
| | pharmaceutically acceptable salt | thereof | |
| | thereof | | |
| 5C* | I-A or pharmaceutically | Gemcitabine or | 10:1 to 90:1 |
| | acceptable salt thereof or I-B or | pharmaceutically acceptable salt | |
| | pharmaceutically acceptable salt | thereof | |
| | thereof | | |
| 6C* | I-A or pharmaceutically | Gemcitabine or | 20:1 to 170:1 |
| | acceptable salt thereof or I-B or | pharmaceutically acceptable salt | |
| | pharmaceutically acceptable salt | thereof | |
| | thereof | | |
| 7C* | I-B or pharmaceutically | Gemcitabine or | 3:1 to 670:1 |
| | acceptable salt thereof | pharmaceutically acceptable salt | |
| | | thereof | |
| 8C* | I-B or pharmaceutically | Gemcitabine or | 10:1 to 190:1 |
| | acceptable salt thereof | pharmaceutically acceptable salt | |
| | | thereof | |
| 9C* | I-B or pharmaceutically | Gemcitabine or | 101: to 170:1 |
| | acceptable salt thereof | pharmaceutically acceptable salt | |
| | | thereof | |
| 10C* | I-B or pharmaceutically | Gemcitabine or | 10:1 to 130:1 |
| | acceptable salt thereof | pharmaceutically acceptable salt | |
| | | thereof | |
| 11C* | I-B or pharmaceutically | Gemcitabine or | 10:1 to 90:1 |
| | acceptable salt thereof | pharmaceutically acceptable salt | |
| | | thereof | |
| 12C* | I-B or pharmaceutically | Gemcitabine or | 20:1 to 170:1 |
| | acceptable salt thereof | pharmaceutically acceptable salt | |
| | | thereof | |
| 13C | I-B as dihydrochloride salt | Gemcitabine hydrochloride | 3:1 to 670:1 |
| 14C | I-B as dihydrochloride salt | Gemcitabine hydrochloride | 10:1 to 190:1 |
| 15C | I-B as dihydrochloride salt | Gemcitabine hydrochloride | 101: to 170:1 |
| 16C | I-B as dihydrochloride salt | Gemcitabine hydrochloride | 10:1 to 130:1 |
| 17C | I-B as dihydrochloride salt | Gemcitabine hydrochloride | 10:1 to 90:1 |
| 18C | I-B as dihydrochloride salt | Gemcitabine hydrochloride | 20:1 to 170:1 |

*The weight:weight ratio for each embodiment is the mole equivalent weight:weight ratio of the indicated weight weight ratios, which are based on CPI as the dihydrochloride salt of the compound of formula I-B and CPII as gemcitabine hydrochloride.

All ratios described in Table B and C above are determined according to the same methodology as given for mole dosage ratios described above, i.e. the ratio of the total weight amount of the doses of the compound of formula II or pharmaceutically acceptable salt thereof to the total weight amount of the doses of the compound of formula I or pharmaceutically acceptable derivative thereof over the respective treatment cycles. Likewise, if a continuous treatment schedule is used for one combination partner and a cyclic treatment schedule is used for the other combination partner then the total weight amount of the doses of the continuous treatment over a period of the same duration of the treatment cycle of the other combination partner is used to determine the weight:weight ratio.

The method of treating neoplastic diseases such as cancer according to the invention may comprise (i) administration of the agent (a) in free or pharmaceutically acceptable salt form and (ii) administration of agent (b) in free or pharmaceutically acceptable salt form simultaneously or sequentially in any order, in jointly therapeutically effective amounts, e.g. in synergistically effective amounts, e.g. in continuous or intermittent dosing schedule corresponding to the amounts described herein. The individual combination partners of the combination of the invention may be administered separately at different times during the course of therapy or concurrently. The invention is therefore to be understood as embracing all such regimens of simultaneous or alternating treatment and the term “administering” is to be interpreted accordingly.

Effective dosages of each of the combination partners employed in the combinations of the invention may vary depending on the particular compound or pharmaceutical composition employed, the mode of administration, the condition being treated, and the severity of the condition being treated. Thus, the dosage regimen of the combination of the invention is selected in accordance with a variety of factors including the route of administration and the renal and hepatic function of the patient.

The optimum ratios, individual and combined dosages, and concentrations of the combination partners of the pharmaceutical combination of the invention that yield efficacy without toxicity are based on the kinetics of the therapeutic agents' availability to target sites. They may be established using routine clinical testing and procedures that are well known in the art and will depend upon a variety of factors, such as the mode of administration, the condition being treated and the severity of the condition being treated, as well as the age, body weight, general health, gender and diet of the individual and other medications the individual is taking.

Likewise, frequency of dosage may vary depending on the compound used and the particular condition to be treated. Patients may generally be monitored for therapeutic effectiveness using assays suitable for the condition being treated, which will be familiar to those of ordinary skill in the art.

When the combination partners, which are employed in the combination of the invention, are applied in the form as marketed as single drugs, their dosage and mode of administration may, in some embodiments, be in accordance with the information provided on the package insert of the respective marketed drugs. In some embodiments the unit dosage forms containing the combination of agents as described herein will contain the amounts of each agent of the combination that are typically administered when the agents are administered alone.

Generally the compound of formula I (e.g. the compound of formula I-A), or derivative thereof (e.g. the compound of formula I-B or pharmaceutically acceptable salt thereof, e.g. the dihydrochloride salt) may be administered orally or intravenously and will be administered at dosages which do not exceed the maximum tolerated dose (MTD) for a particular mode of administration and indication, as determined in a clinical dose escalation study.

When administered orally the dosage of the compound of formula I-B as the dihydrochloride salt per day on days when administered may be e.g. in the range of about 1 mg to about 30 mg (e.g. 1 mg to 30 mg), e.g. in the range of about 2 mg to about 20 mg (e.g. 2 mg to 20 mg), e.g. in the range of about 4 mg to about 20 mg (e.g. 4 mg to 20 mg), e.g. in the range of about 8 mg to about 20 mg (e.g. 8 mg to 20 mg), or in any single amount within these ranges (e.g. 4 mg, 8 mg, 12 mg or 16 mg)). For example the dosage per day on days when administered may be at least about 1 mg, e.g. at least about 2 mg, e.g. at least about 4 mg, e.g. at least about 8 mg, e.g. up to about 50 mg, e.g. up to about 30 mg, e.g. up to about 20 mg. When a different compound of formula I is administered, e.g. the compound of formula I-A in free form or the compound of formula I-B as a pharmaceutically acceptable salt other than the dihydrochloride salt, then the corresponding dosages amounts to give the same number of moles are administered based on the respective molecular weights.

When administered orally the compound of formula I (e.g. the compound of formula I-A), or derivative thereof (e.g. the compound of formula I-B or pharmaceutically acceptable salt thereof, e.g. the dihydrochloride salt) may be administered according to a continuous treatment schedule or a cyclic treatment schedule. Administration may be more than once per day (e.g. twice per day) if needed or desired and the dosage per administration is reduced accordingly so that the dosage on a given day remains within the specified limits. In one embodiment administration is according to a continuous treatment schedule with one dose per day for as long as needed. In one embodiment administration is according to a continuous treatment schedule with two doses per day for as long as needed.

When administered intravenously the dose of the compound of formula I-B as the dihydrochloride salt per week during weeks when administered may be e.g. in the range of about 1 mg/m² to about 160 mg/m² (e.g. 1 mg/m² to 160 mg/m²), e.g. in the range of about 15 mg/m² to about 100 mg/m² (e.g. 15 mg/m² to 100 mg/m²), e.g. in the range of about 30 mg/m² to about 100 mg/m² (e.g. 30 mg/m² to 100 mg/m²), e.g. in the range of about 30 mg/m² to about 70 mg/m² (e.g. 30 mg/m² to 70 mg/m²), or in any single amount within these ranges (e.g. 30 mg/m², 45 mg/m², 70 mg/m² or 90 mg/m²)). For example the dose per week during weeks when administered may be e.g. at least 1 mg/m², e.g. at least about 10 mg/m², e.g. at least about 15 mg/m², e.g. at least about 30 mg/m², e.g. up to about 160 mg/m², e.g. up to about 100 mg/m², e.g. up to about 70 mg/m². Likewise as above, when a different compound of formula I is administered, e.g. the compound of formula I-A in free form or the compound of formula I-B as a pharmaceutically acceptable salt other than the dihydrochloride salt, then the corresponding dosages amounts to give the same number of moles is administered based on the respective molecular weights.

When administered intravenously the compound of formula I (e.g. the compound of formula I-A), or derivative thereof (e.g. the compound of formula I-B or pharmaceutically acceptable salt thereof, e.g. the dihydrochloride salt) may be administered once per week or more than once per week, e.g. twice or three times per week. The intravenous dose may be over a period as long as needed, e.g. over a period of about 1 to about 96 hours, e.g. about 40 to about 80 hours, e.g. about 72 hours, e.g. about 40 hours to about 60 hours. In one embodiment the dose may be over a period of about 48 hours. In another embodiment the dose may be over a period of about 60 hours. In another embodiment the dose may be over a period of about 72 hours. Such intravenous administration may utilise a continuous infusion pump or other intravenous administration device.

In another embodiment the duration of administration may be e.g. a period of about 1 to about 4 hours, e.g. about 2 hours.

In one embodiment the compound of formula I or pharmaceutically acceptable derivative thereof is administered according to a 21-day treatment cycle with two days of dosing, e.g. initiated on days 1 and 8. In another embodiment the compound of formula I or pharmaceutically acceptable derivative thereof is administered according to a 28-day treatment cycle with three days of dosing, e.g. initiated on days 1, 8 and 15. In another embodiment the compound of formula I or pharmaceutically acceptable derivative thereof is administered according to a 28-day treatment cycle with two days of dosing, e.g. initiated on days 1 and 15.

The dose of the compound of formula II as gemcitabine hydrochloride per week during weeks when administered may be e.g. in the range of about 100 mg/m² to about 1400 mg/m², (e.g. 100 mg/m² to 1400 mg/m²), e.g. in the range of about 200 mg/m² to about 1300 mg/m², (e.g. 200 to 1300 mg/m²), or in any single amount within these ranges (e.g. 250 mg/m², 1000 mg/m² or 1250 mg/m²). For example the dose per week during weeks when administered may be e.g. at least about 100 mg/m², e.g. at least about 200 mg/m², e.g. up to about 1400 mg/m², e.g. up to about 1300 mg/m². When a salt of the compound of formula II other than the hydrochloride salt is administered, then the corresponding dosage amounts to give the same number of moles is administered based on the molecular weight of Gemcitabine hydrochloride (given above).

The compound of formula II or pharmaceutically acceptable salt thereof can be administered as a single dose e.g. once per day, once per week, once every two weeks or once per month, or more than one dose can be administered per day, per week, per two weeks, or per month. The intravenous dose may be over a period for as long as needed, e.g. over a period of about 1 minute to about 60 minutes, e.g. over a period of about 20 to 40 minutes, e.g. about 30 minutes. In one embodiment the compound of formula II or pharmaceutically acceptable salt thereof is administered according to a 21-day treatment cycle with 2 days of dosing, e.g. initiated on days 1 and 8. In another embodiment the compound of formula I or pharmaceutically acceptable derivative thereof is administered according to a 28-day treatment cycle with three days of dosing, e.g. initiated on days 1, 8 and 15. In another embodiment the compound of formula I or pharmaceutically acceptable derivative thereof is administered according to a 28-day treatment cycle with four days of dosing, e.g. initiated on days 1, 8, 15 and 21.

More specifically, a possible dose of the compound of formula II or pharmaceutically acceptable salt (e.g. gemcitabine hydrochloride) is 1000 mg/m² administered intravenously over 30 minutes on days 1 and 8 of a 21-day cycle. This dosage regime has been approved for the treatment of ovarian cancer.

A further possible dose of the compound of formula II or pharmaceutically acceptable salt (e.g. gemcitabine hydrochloride) is 1250 mg/m² administered intravenously over 30 minutes on days 1 and 8 of a 21-day cycle. This dosage regime has been approved for the treatment of breast cancer. A further possible dose of the compound of formula II or pharmaceutically acceptable salt (e.g. gemcitabine hydrochloride) is 1000 mg/m² administered intravenously over 30 minutes on days 1, 8 and 15 of a 28-day cycle. This dosage regime has been approved for the treatment of non-small cell lung cancer. A further possible dose of the compound of formula II or pharmaceutically acceptable salt (e.g. gemcitabine hydrochloride) is 1000 mg/m² administered intravenously over 30 minutes once weekly for 7 weeks, then one rest week, then once weekly for three weeks of each 28-day cycle. This dosage regime has been approved for the treatment of pancreatic cancer. A further possible dose of the compound of formula II or pharmaceutically acceptable salt (e.g. gemcitabine hydrochloride) is 250 mg/m² administered intravenously over 30 minutes once weekly. This dosage regime has been found to be effective for treatment of pancreatic cancer (Sakamoto et al. “Comparison of standard-dose and low-dose gemcitabine regimens in pancreatic adenocarcinoma patients: a prospective randomized trial.” J Gastroenterol, 41(1), pages 70-76, 2006.

The compound of formula I (e.g. the compound of formula I-A or pharmaceutically acceptable salt thereof) or derivative thereof (e.g. the compound of formula I-B or pharmaceutically acceptable salt thereof) and compound of formula II or pharmaceutically acceptable salt thereof (e.g. gemcitabine hydrochloride) compositions can be administered to a patient substantially simultaneously or sequentially and in either order (e.g. administration of the compound of formula I or derivative thereof prior to the compound of formula II or pharmaceutically acceptable salt thereof, or vice versa).

In one embodiment the compound of formula I-B as the dihydrochloride salt is administered to a patient orally, e.g. wherein the compound is administered every day and the dose per day is about 2 mg to about 30 mg, e.g. about 2 mg to about 20 mg, e.g. about 4 mg to about 20 mg, e.g. about 8 mg to about 20 mg), while gemcitabine hydrochloride is administered to a patient by intravenous infusion wherein the dose per week during weeks when administered is e.g. about 100 mg/m² to 1400 mg/m², e.g. about 200 to about 1300 mg/m², e.g. 250 mg/m², 1000 mg/m² or 1250 mg/m² e.g. over about 1 minute to about 60 minutes, e.g. over about 20 minutes to about 40 minutes, e.g. about 30 minutes e.g. with 2 days of dosing in a 21-day treatment cycle, e.g. initiated on days 1 and 8, or e.g. three days of dosing in a 28-day treatment cycle, e.g. initiated on days 1, 8 or 15 or four days of dosing in a 28-day treatment cycle, e.g. initiated on days 1, 8, 15 and 21 (in this case usually with a lower dose, e.g. 250 mg/m²). This course of treatment can be repeated (e.g. 1 to 8 times or may be open-ended), as determined to be tolerable and effective by those of skill in the art.

In another embodiment the compound of formula I-B as the dihydrochloride salt) is administered to a patient by intravenous infusion, e.g. wherein the dose per week during weeks when administered is about 15 mg/m² to about 160 mg/m², e.g. about 15 mg/m² to about 100 mg/m², e.g. about 30 mg/m² to about 100 mg/m², e.g. about 30 mg/m² to about 70 mg/m², e.g. over a period of about 24 to about 72 hours, with e.g. two days of dosing in a 21-day treatment cycle, e.g. initiated on days 1 and 8, or e.g. two days of dosing in a 28-day treatment cycle, e.g. initiated on days 1 and 15, or e.g. three days of dosing in a 28-day treatment cycle, e.g. on day 1, 8 and 15, while gemcitabine hydrochloride is administered to a patient by intravenous infusion wherein the dose per week during weeks when administered is e.g. about 100 mg/m² to 1400 mg/m² e.g. about 200 to about 1300 mg/m², e.g. 250 mg/2, 1000 mg/m² or 1250 mg/m² e.g. over about 1 minute to about 60 minutes, e.g. over about 20 minutes to about 40 minutes, e.g. about 30 minutes e.g. with 2 days of dosing in a 21-day treatment cycle, e.g. initiated on days 1 and 8, or e.g. three days of dosing in a 28-day treatment cycle, e.g. initiated on days 1, 8 or 15 or four days of dosing in a 28-day treatment cycle, e.g. initiated on days 1, 8, 15 and 21 (in this case usually with a lower dose, e.g. 250 mg/2). This course of treatment can be repeated (e.g. 1 to 8 times or may be open-ended), as determined to be tolerable and effective by those of skill in the art.

Additional embodiments of the invention are shown in Table D below.

TABLE D

| | Combination | Combination | Dosage CPI (depicted above) |
|---|---|---|---|
| Embodiment | partner I (CPI) | partner II (CPII) | Dosage CPII (depicted below) |
| 1D* | I-A or | Gemcitabine or | oral, 2 to 30 mg (dose per day on days when |
| | pharmaceutically | pharmaceutically | administered) |
| | acceptable salt thereof | acceptable salt | IV, 100 mg/m² to 1400 mg/m² (dose per week in |
| | or I-B or | thereof | weeks when administered) |
| | pharmaceutically | | |
| | acceptable salt thereof | | |
| 2D* | I-A or | Gemcitabine or | oral, 4 to 20 mg (dose per day), dose every day |
| | pharmaceutically | pharmaceutically | IV, 200 mg/m² to 1300 mg/m² (dose per week in |
| | acceptable salt thereof | acceptable salt | weeks when administered), 2 doses per 21-day |
| | I-B or | thereof | treatment cycle e.g. initiated on days 1 and 8 |
| | pharmaceutically | | |
| | acceptable salt thereof | | |
| 3D* | I-A or | Gemcitabine or | oral, 4 to 20 mg (dose per day), dose every day |
| | pharmaceutically | pharmaceutically | IV, 200 mg/m² to 1300 mg/m² (dose per week dose |
| | acceptable salt thereof | acceptable salt | in weeks when administered), 3 doses per 28-day |
| | I-B or | thereof | treatment cycle e.g. initiated on days 1, 8 and 15 |
| | pharmaceutically | | |
| | acceptable salt thereof | | |
| 4D* | I-A or | Gemcitabine or | oral, 8 to 20 mg (dose per day), dose every day |
| | pharmaceutically | pharmaceutically | IV, 200 mg/m² to 1300 mg/m² (dose per week in |
| | acceptable salt thereof | acceptable salt | weeks when administered), 2 doses per 21-day |
| | I-B or | thereof | treatment cycle e.g. initiated on days 1 and 8 |
| | pharmaceutically | | |
| | acceptable salt thereof | | |
| 5D* | I-A or | Gemcitabine or | oral, 8 to 20 mg (dose per day), dose every day |
| | pharmaceutically | pharmaceutically | IV, 200 mg/m² to 1300 mg/m² (dose per week dose |
| | acceptable salt thereof | acceptable salt | in weeks when administered), 3 doses per 28-day |
| | I-B or | thereof | treatment cycle e.g. initiated on days 1, 8 and 15 |
| | pharmaceutically | | |
| | acceptable salt thereof | | |
| 6D* | I-B or | Gemcitabine or | oral, 2 to 30 mg (dose per day on days when |
| | pharmaceutically | pharmaceutically | administered) |
| | acceptable salt thereof | acceptable salt | IV, 100 mg/m² to 1400 mg/m² (dose per week in |
| | | thereof | weeks when administered) |
| 7D* | I-B or | Gemcitabine or | oral, 4 to 20 mg (dose per day), dose every day |
| | pharmaceutically | pharmaceutically | IV, 200 mg/m² to 1300 mg/m² (dose per week in |
| | acceptable salt thereof | acceptable salt | weeks when administered), 2 doses per 21-day |
| | | thereof | treatment cycle e.g. initiated on days 1 and 8 |
| 8D* | I-B or | Gemcitabine or | oral, 4 to 20 mg (dose per day), dose every day |
| | pharmaceutically | pharmaceutically | IV, 200 mg/m² to 1300 mg/m² (dose per week dose |
| | acceptable salt thereof | acceptable salt | in weeks when administered), 3 doses per 28-day |
| | | thereof | treatment cycle e.g. initiated on days 1, 8 and 15 |
| 9D* | I-B or | Gemcitabine or | oral, 8 to 20 mg (dose per day), dose every day |
| | pharmaceutically | pharmaceutically | IV, 200 mg/m² to 1300 mg/m² (dose per week in |
| | acceptable salt thereof | acceptable salt | weeks when administered), 2 doses per 21-day |
| | | thereof | treatment cycle e.g. initiated on days 1 and 8 |
| 10D* | I-B or | Gemcitabine or | oral, 8 to 20 mg (dose per day), dose every day |
| | pharmaceutically | pharmaceutically | IV, 200 mg/m² to 1300 mg/m² (dose per week dose |
| | acceptable salt thereof | acceptable salt | in weeks when administered), 3 doses per 28-day |
| | | thereof | treatment cycle e.g. initiated on days 1, 8 and 15 |
| 11D* | I-B as dihydrochloride | Gemcitabine | oral, 2 to 30 mg (dose per day on days when |
| | salt | hydrochloride | administered) |
| | | | IV, 100 mg/m² to 1400 mg/m² (dose per week in |
| | | | weeks when administered) |
| 12D* | I-B as dihydrochloride | Gemcitabine | oral, 4 to 20 mg (dose per day), dose every day |
| | salt | hydrochloride | IV, 200 mg/m² to 1300 mg/m² (dose per week in |
| | | | weeks when administered), 2 doses per 21-day |
| | | | treatment cycle e.g. initiated on days 1 and 8 |
| 13D* | I-B as dihydrochloride | Gemcitabine | oral, 4 to 20 mg (dose per day), dose every day |
| | salt | hydrochloride | IV, 200 mg/m² to 1300 mg/m² (dose per week dose |
| | | | in weeks when administered), 3 doses per 28-day |
| | | | treatment cycle e.g. initiated on days 1, 8 and 15 |
| 14D* | I-B as dihydrochloride | Gemcitabine | oral, 8 to 20 mg (dose per day), dose every day |
| | salt | hydrochloride | IV, 200 mg/m² to 1300 mg/m² (dose per week in |
| | | | weeks when administered), 2 doses per 21-day |
| | | | treatment cycle e.g. initiated on days 1 and 8 |
| 15D* | I-B as dihydrochloride | Gemcitabine | oral, 8 to 20 mg (dose per day), dose every day |
| | salt | hydrochloride | IV, 200 mg/m² to 1300 mg/m² (dose per week dose |
| | | | in weeks when administered), 3 doses per 28-day |
| | | | treatment cycle e.g. initiated on days 1, 8 and 15 |
| 16D* | I-A or | Gemcitabine or | IV, 30 to 100 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | I-B or | thereof | IV, 200 mg/m² to 1300 mg/m² (accumulated dose |
| | pharmaceutically | | per week in weeks when administered), 2 doses per |
| | acceptable salt thereof | | 21-day treatment cycle, e.g. initiated on days 1 and |
| | | | 8 |
| 17D* | I-A or | Gemcitabine or | IV, 30 to 100 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | I-B or | thereof | IV, 200 mg/m² to 1300 mg/m² (dose per week in |
| | pharmaceutically | | weeks when administered), 2 doses per 21-day |
| | acceptable salt thereof | | treatment cycle e.g. initiated on days 1 and 8 |
| 18D* | I-A or | Gemcitabine or | IV, 30 to 100 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | I-B or | thereof | IV, 200 mg/m² to 1300 mg/m² (dose per week in |
| | pharmaceutically | | weeks when administered), 3 doses per 28-day |
| | acceptable salt thereof | | treatment cycle e.g. initiated on days 1, 8 and 15 |
| 19D* | I-A or | Gemcitabine or | IV, 30 to 100 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | I-B or | thereof | IV, 200 mg/m² to 1300 mg/m² (dose per week in |
| | pharmaceutically | | weeks when administered), 3 doses per 28-day |
| | acceptable salt thereof | | treatment cycle e.g. initiated on days 1, 8 and 15 |
| 20D* | I-A or | Gemcitabine or | IV, 30 to 70 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | I-B or | thereof | IV, 200 mg/m² to 1300 mg/m² (accumulated dose |
| | pharmaceutically | | per week in weeks when administered), 2 doses per |
| | acceptable salt thereof | | 21-day treatment cycle, e.g. initiated on days 1 and |
| | | | 8 |
| 21D* | I-A or | Gemcitabine or | IV, 30 to 70 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | I-B or | thereof | IV, 200 mg/m² to 1300 mg/m² (dose per week in |
| | pharmaceutically | | weeks when administered), 2 doses per 21-day |
| | acceptable salt thereof | | treatment cycle e.g. initiated on days 1 and 8 |
| 22D* | I-A or | Gemcitabine or | IV, 30 to 70 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | I-B or | thereof | IV, 0.3 to 1.5 mg/m² (dose per week in weeks when |
| | pharmaceutically | | administered), 3 doses per 28-day treatment cycle |
| | acceptable salt thereof | | e.g. initiated on days 1, 8 and 15 |
| 23D* | I-A or | Gemcitabine or | IV, 30 to 70 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | I-B or | thereof | IV, 200 mg/m² to 1300 mg/m² (dose per week in |
| | pharmaceutically | | weeks when administered), 3 doses per 28-day |
| | acceptable salt thereof | | treatment cycle e.g. initiated on days 1, 8 and 15 |
| 24D* | I-B or | Gemcitabine or | IV, 30 to 100 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | | thereof | IV, 200 mg/m² to 1300 mg/m² (accumulated dose |
| | | | per week in weeks when administered), 2 doses per |
| | | | 21-day treatment cycle, e.g. initiated on days 1 and |
| | | | 8 |
| 25D* | I-B or | Gemcitabine or | IV, 30 to 100 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | | thereof | IV, 200 mg/m² to 1300 mg/m² (dose per week in |
| | | | weeks when administered), 2 doses per 21-day |
| | | | treatment cycle e.g. initiated on days 1 and 8 |
| 26D* | I-B or | Gemcitabine or | IV, 30 to 100 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | | thereof | IV, 200 mg/m² to 1300 mg/m² (dose per week in |
| | | | weeks when administered), 3 doses per 28-day |
| | | | treatment cycle e.g. initiated on days , 81 and 15 |
| 27D* | I-B or | Gemcitabine or | IV, 30 to 100 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | | thereof | IV, 200 mg/m² to 1300 mg/m² (dose per week in |
| | | | weeks when administered), 3 doses per 28-day |
| | | | treatment cycle e.g. initiated on days 1, 8 and 15 |
| 28D* | I-B or | Gemcitabine or | IV, 30 to 70 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | | thereof | IV, 200 mg/m² to 1300 mg/m² (accumulated dose |
| | | | per week in weeks when administered), 2 doses per |
| | | | 21-day treatment cycle, e.g. initiated on days 1 and |
| | | | 8 |
| 29D* | I-B or | Gemcitabine or | IV, 30 to 70 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | | thereof | IV, 200 mg/m² to 1300 mg/m² (dose per week in |
| | | | weeks when administered), 2 doses per 21-day |
| | | | treatment cycle e.g. initiated on days 1 and 8 |
| 30D* | I-B or | Gemcitabine or | IV, 30 to 70 mg/m² (dose per week in weeks when |
| | pharmaceutically | pharmaceutically | administered), 2 doses per 21-day treatment cycle, |
| | acceptable salt thereof | acceptable salt | e.g. initiated on days 1 and 8 |
| | | thereof | IV, 200 mg/m² to 1300 mg/m² (dose per week in |
| | | | weeks when administered), 3 doses per 28-day |
| | | | treatment cycle e.g. initiated on days 1, 8 and 15 |
| 31D* | I-B or | Gemcitabine or | IV, 30 to 70 mg/m² (dose per week in weeks when |
| | pharmaceutically | pharmaceutically | administered), 3 doses per 28-day treatment cycle, |
| | acceptable salt thereof | acceptable salt | e.g. initiated on days 1, 8 and 15 |
| | | thereof | IV, 200 mg/m² to 1300 mg/m² (dose per week in |
| | | | weeks when administered), 3 doses per 28-day |
| | | | treatment cycle e.g. initiated on days 1, 8 and 15 |
| 32D* | I-B as dihydrochloride | Gemcitabine | IV, 30 to 100 mg/m² (dose per week in weeks when |
| | salt | hydrochloride | administered), 2 doses per 21-day treatment cycle, |
| | | | e.g. initiated on days 1 and 8 |
| | | | IV, 200 mg/m² to 1300 mg/m² (accumulated dose |
| | | | per week in weeks when administered), 2 doses per |
| | | | 21-day treatment cycle, e.g. initiated on days 1 and |
| | | | 8 |
| 33D* | I-B as dihydrochloride | Gemcitabine | IV, 30 to 100 mg/m² (dose per week in weeks when |
| | salt | hydrochloride | administered), 3 doses ng per 28-day treatment |
| | | | cycle, e.g. initiated on days 1, 8 and 15 |
| | | | IV, 200 mg/m² to 1300 mg/m² (dose per week in |
| | | | weeks when administered), 2 doses per 21-day |
| | | | treatment cycle e.g. initiated on days 1 and 8 |
| 34D* | I-B as dihydrochloride | Gemcitabine | IV, 30 to 100 mg/m² (dose per week in weeks when |
| | salt | hydrochloride | administered), 2 doses per 21-day treatment cycle, |
| | | | e.g. initiated on days 1 and 8 |
| | | | IV, 200 mg/m² to 1300 mg/m² (dose per week in |
| | | | weeks when administered), 3 doses per 28-day |
| | | | treatment cycle e.g. initiated on days 1, 8 and 15 |
| 35D* | I-B as dihydrochloride | Gemcitabine | IV, 30 to 100 mg/m² (dose per week in weeks when |
| | salt | hydrochloride | administered), 3 doses per 28-day treatment cycle, |
| | | | e.g. initiated on days 1, 8 and 15 |
| | | | IV, 200 mg/m² to 1300 mg/m² (dose per week in |
| | | | weeks when administered), 3 doses per 28-day |
| | | | treatment cycle e.g. initiated on days 1, 8 and 15 |
| 36D* | I-B as dihydrochloride | Gemcitabine | IV, 30 to 70 mg/m² (dose per week in weeks when |
| | salt | hydrochloride | administered), 2 doses per 21-day treatment cycle, |
| | | | e.g. initiated on days 1 and 8 |
| | | | IV, 200 mg/m² to 1300 mg/m² (accumulated dose |
| | | | per week in weeks when administered), 2 doses per |
| | | | 21-day treatment cycle, e.g. initiated on days 1 and 8 |
| 37D* | I-B as dihydrochloride | Gemcitabine | IV, 30 to 70 mg/m² (dose per week in weeks when |
| | salt | hydrochloride | administered), 3 doses ng per 28-day treatment |
| | | | cycle, e.g. initiated on days 1, 8 and 15 |
| | | | IV, 200 mg/m² to 1300 mg/m² (dose per week in |
| | | | weeks when administered), 2 doses per 21-day |
| | | | treatment cycle e.g. initiated on days 1 and 8 |
| 38D* | I-B as dihydrochloride | Gemcitabine | IV, 30 to 70 mg/m² (dose per week in weeks when |
| | salt | hydrochloride | administered), 2 doses per 21-day treatment cycle, |
| | | | e.g. initiated on days 1 and 8 |
| | | | IV, 200 mg/m² to 1300 mg/m² (dose per week in |
| | | | weeks when administered), 3 doses per 28-day |
| | | | treatment cycle e.g. initiated on days 1, 8 and 15 |
| 39D* | I-B as dihydrochloride | Gemcitabine | IV, 30 to 70 mg/m² (dose per week in weeks when |
| | salt | hydrochloride | administered), 3 doses per 28-day treatment cycle, |
| | | | e.g. initiated on days 1, 8 and 15 |
| | | | IV, 200 mg/m² to 1300 mg/m² (dose per week in |
| | | | weeks when administered), 3 doses per 28-day |
| | | | treatment cycle e.g. initiated on days 1, 8 and 15 |

*The applicable weight range for each embodiment is the mole equivalent weight range of the indicated weight range, which is based on combination partner I as the dihydrochloride salt of the compound of formula I-B and combination partner II as Gemcitabine hydrochloride.

Formulations

The combination of the invention may be formulated as pharmaceutical compositions for non-parenteral administration, such as nasal, buccal, rectal, pulmonary, vaginal, sublingual, topical, transdermal, ophthalmic, otic or, especially, for oral administration, e.g. in the form of oral solid dosage forms, e.g. granules, pellets, powders, tablets, film or sugar coated tablets, effervescent tablets, hard and soft gelatin or HPMC capsules, coated as applicable, orally disintegrating tablets, oral solutions, lipid emulsions or suspensions, or for parenteral administration, such as intravenous, intramuscular, or subcutaneous, intrathecal, intradermal or epidural administration, to mammals, especially humans, e.g. in the form of solutions, lipid emulsions or suspensions containing microparticles or nanoparticles. The compositions may comprise the active ingredient(s) alone or, preferably, together with a pharmaceutically acceptable carrier.

The pharmaceutical compositions can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc. Suitable excipients for parenteral formulations are e.g. water, alcohols, polyols, glycerol, vegetable oils, lecithin, surfactants etc. Moreover, the pharmaceutical preparations can contain preservatives, solubilizers, stabilizers, wetting agents, emulsifiers, sweeteners, colorants, flavourants, salts for varying the osmotic pressure, buffers, masking agents or antioxidants. They can also contain other therapeutically valuable substances.

In addition pharmaceutical compositions used in the invention optionally include buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the pharmaceutical compositions contain a pharmaceutically acceptable preservative. In some embodiments the preservative concentration ranges from 0.1 to 2.0 percent, typically v/v. Suitable preservatives include those known in the pharmaceutical arts, such as benzyl alcohol, phenol, m-cresol, methylparaben, and propylparaben.

An example of an oral composition of the compound of formula I, e.g. the compound formula I-B in the form of its dihydrochloride salt, includes but is not limited to HPMC capsules containing 1 mg active ingredient, 98 mg of mannitol and 1 mg magnesium stearate, or 5 mg active ingredient, 94 mg mannitol and 1 mg magnesium stearate.

For intravenous administration of the compound of formula I-B, e.g. the compound of formula I-B in the form of its dihydrochloride salt, the compound of formula I or derivative thereof may be provided in powder (e.g. lyophilized) form and reconstituted with a suitable diluent, e.g. saline solution or Ringer lactate solution, immediately prior to administration. The active ingredient may be initially reconstituted with saline solution or Ringer lactate solution and then diluted to the required concentration with Ringer lactate solution.

Gemcitabine is typically provided in powder form for reconstitution with saline solution for intravenous administration. In one example gemcitabine hydrochloride is reconstituted in 0.9 weight percent sodium chloride in water for injection (0.9% Sodium Chloride Injection USP).

The pharmaceutical composition may contain, from about 0.1 percent to about 99.9 percent, preferably from about 1 percent to about 60 percent, of the therapeutic agent(s)

Kits

The invention also provides pharmaceutical products such as kits which may include a container with the compound of formula I or derivative thereof (e.g. the compound of formula I-A or pharmaceutically acceptable salt thereof or the compound of formula I-B or pharmaceutically acceptable salt thereof) and/or a container with the compound of formula II or pharmaceutically acceptable salt thereof (e.g. gemcitabine hydrochloride). The active ingredients in such kits can be provided in amounts sufficient to treat a neoplastic disease such as cancer in a patient in need thereof (e.g. amounts sufficient for a single administration or for multiple administrations). The kits can thus include multiple containers which each include pharmaceutically effective amounts of the active ingredients. Optionally, instruments and/or devices necessary for administering the pharmaceutical composition(s) can also be included in the kits. Furthermore, the kits can include additional components, such as instructions or administration schedules, for treating a patient with cancer with the combinations of the invention.

Accordingly, in a further aspect the invention provides a pharmaceutical product such as a kit e.g. for use in treating a neoplastic disease such as cancer, the pharmaceutical product comprising the pharmaceutical combination of the invention, wherein component (a) and component (b) are provided as separate dosage units. In one embodiment the kit further comprises instructions for simultaneous, separate or sequential administration thereof for use in the treatment of a neoplastic disease, in particular a cancer.

Additional Therapeutics

The combination of the invention may used alone in the treatment of the medical conditions described herein. It is also contemplated that the combination is used together with a surgical procedure (for example to remove or reduce the size of a tumour), radiation therapy, ablation therapy and/or one or more therapeutic agents other than a compound of the formula I or formula II. Examples of anti-cancer agents that can be used together with the combination of the invention include but are not limited to chemotherapy (cytotoxic therapy), kinase inhibitors, endocrine therapy, biologics, immunotherapy, or a combination of these.

Gemcitabine is approved for use in combination with nab-paclitaxel for the treatment of metastatic pancreatic cancer. The combinations of the invention may be used in combination with nab-paclitaxel, e.g. for the treatment of pancreatic cancer. Gemcitabine is approved for use in combination with carboplatin for the treatment of ovarian cancer. The combinations of the invention may be used in combination with carboplatin, e.g. for the treatment of ovarian cancer. Gemcitabine is approved for use in combination with paclitaxel for metastatic breast cancer. The combinations of the invention may be used in combination with paclitaxel e.g. for the treatment of breast cancer, in particular metastatic breast cancer. Gemcitabine is approved for use in combination with cisplatin for the treatment of non-small cell lung cancer. The combinations of the invention may be used in combination with cisplatin, e.g. for the treatment of non-small cell lung cancer.

All aspects and embodiments of the invention described herein may be combined in any combination where possible.

For the avoidance of doubt, where ranges are mentioned (e.g. “in the range of . . . ”) the end points of the range are also included in the range.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### Examples

In Vivo PAXF1657 Tumor Xenografts in Mice

Animal Housing

Animals were housed in individually ventilated cages (TECNIPLAST Sealsafe™-IVC-System, TECNIPLAST, Hohenpeissenberg, Germany), depending on group size, either in type III or type II long cages. They were kept under a 14 L:10D artificial light cycle. The temperature inside the cages was maintained at 25±1° C. with a relative humidity of 40-70% and an air change (AC) rate in the cage of 60-65 AC/hour. Dust-free bedding consisting of aspen wood chips with approximate dimensions of 5×5×1 mm (ABEDD®-LAB & VET Service GmbH, Vienna, Austria, Product Code: LTE E-001) and additional nesting material were used. The cages including the bedding and the nesting material were changed weekly. The animals were fed autoclaved Teklad Global 19% Protein Extruded Diet (T.2019S.12) from Envigo RMS SARL and had access to sterile filtered and acidified (pH 2.5) tap water that was changed twice weekly. Feed and water were provided ad libitum. All materials were autoclaved prior to use. Animals were provided with a nutrient fortified water gel (DietGel® Recovery from ClearH₂O, Maine, USA) from day 0 to day 45, which was changed every other day.

Tumor Xenografts

The tumor xenografts are derived from surgical specimens from cancer patients. Following excision at surgery, tumor pieces were subcutaneously implanted into immunodeficient mice and are therefore referred to as patient tumor explants passaged subcutaneously in nude mice or as patient-derived tumor xenografts (PDX). Establishment and characterization of the PDXs was performed following their primary implantation into immunodeficient mice (passage 1). The tumor xenografts were passaged until establishment of a stable growth pattern. At that point, master stocks of early passage PDXs were frozen in liquid nitrogen. Usually, a particular stock batch is only used for a limited number of further passages.

Tumor Implantation and Tumor Growth Measurement

Tumor fragments for implantation were obtained from xenografts in serial passage in female NMRI nu/nu mice (NMRI-Foxn1nu). After removal from donor mice, tumors were cut into fragments (3-4 mm edge length) and placed in PBS containing 10% penicillin/streptomycin. Recipient female mice (10 weeks old NMRI-Foxn1nu animals) were anesthetized by inhalation of isoflurane and received unilateral tumor implants of PAXF1657 tumors subcutaneously in the flank.

Mice and tumor implants were monitored daily until solid tumor growth was detectable in a sufficient number of animals. At randomization, the volume of growing tumors was determined. Tumors were measured (mm) in two dimensions using calipers, and volume was calculated using the formula: Tumor Volume (TV) (mm³)=(width²×length)/2. Animals fulfilling the randomization criteria (i.e. bearing tumors of 50-250 mm³, preferably 80-200 mm³) were then distributed into experimental groups, aiming at comparable median and mean group tumor volumes. Sixteen days after tumor cell implantation, on Day 0 of the study, mice were randomized into groups of 10 animals, with group median tumor volumes of 106.6-109.2 mm³ and body weights of 25.3-27.0 g.

Preparation of BAL101553 and Gemcitabine Dosing Solutions

The solution of BAL101553 with a concentration of 1.6 mg/ml for dosing at 16 mg/kg and as stock solution for the preparation of the other dosing solutions was prepared at the beginning of the study by dissolving 595.2 mg of the provided powder in 300 ml vehicle (stirring/vortexing/sonicating if necessary). The acidity of this solution was adjusted to pH 5 with a 1% sodium acetate solution; afterwards the total volume was adjusted to 372 ml by addition of the vehicle (99.7% (v/v) saline solution (0.9% (w/v) NaCl), 0.3% (v/v) sodium acetate, pH 5). The stock solution was sterile filtered and aliquoted into vials of each 12 ml that were stored at −20° C. One vial was thawed on each dosing day. For the 16 mg/kg dose, the solution was applied undiluted; for 8 mg/kg dose, one volume of the stock solution was diluted with one volume vehicle prior to application; and for 10 mg/kg dose, one volume of the stock solution was diluted with 0.6 volumes vehicle

The Gemcitabine dosing solution at a concentration of 24 mg/ml for dosing at 240 mg/kg was prepared once per week on the dosing day by dissolving 591.89 mg of Gemcitabine-HCl (Gemedac®, Medac Germany) corresponding to 264 mg active pharmaceutical ingredient in 11 ml of the vehicle (0.9% (w/v) NaCl). The dosing solution was administered in a dose volume of 10 ml/kg.

Treatment

Groups of mice (n=10) bearing PAXF1657 tumors were dosed as outlined in Table 1. All doses were adjusted to 10 mL/kg. The control groups received oral doses of vehicle (pH 5 buffered 0.9% NaCl solution). Gemcitabine was administered i.v. and BAL101553 orally. When Gemcitabine and BAL0101553 were dosed in combination, Gemcitabine was administered first followed by a delay of 4 h for the BAL0101553 dosing.

Determination of Body Weight, Antitumor Activity, Tumor Regression and Tumor Free Survival Rates (Cures)

Body weights and tumor volumes were recorded twice weekly until the end of the on day 60. These parameters were analyzed and graphically represented by using GraphPad-Prism™ 7 for Windows according to standard procedure. Tumor growth regression was defined as TV (at day 21)−TV (day 0)<0. Day 21 was chosen since most of the control animals reached the maximum tolerated tumor volume on day 21 and therefore had to be sacrificed according to animal health guidelines. Tumor free animals at the end of the study time (day 60) were assessed for residual PAXF1657 tumor cells by pathological methods. Skin und subcutaneous tissue including the tumor cell inoculation site from the flank region were resected, preserved in formalin and embedded in paraffin. Sections of approximately 5 μm were mounted on glass slides and stained with hematoxylin/eosin and were evaluated by a trained pathologist, to identify residual tumor cells at the site of injection, and were being reported as positive for the existence of residual tumor cells or as negative for a complete lack of any evidence of residual tumor cells.

LC-MS/MS Method for the Detection of BAL27862, BAL101553 and Gemcitabine Concentrations in Mouse Plasma

Blood samples from tumor bearing mice treated either with a) BAL101553 at 8 mg/kg oral, b) with Gemcitabine 240 mg/kg i.v. or c) with the combination of BAL101553 10 mg/kg oral and Gemcitabine 240 mg/kg i.v. and d) vehicle control treated accordingly, were collected into K₂EDTA tubes at 0.17, 0.5, 1, 3, 8 and 24 h post dosing and immediately processed for plasma and then stored at −80° C. until analysis. From each mouse, two blood samples were taken, first by mandibular bleeding for an early time point and by terminal bleeding for a later time point. For normalization, 25 μL of control mouse plasma samples were mixed with 75 μL of internal standard solution (0.5 μg/mL of BAL27862-d7 and BAL101553-d7 in acetonitrile). The samples were centrifuged and the supernatants were transferred into new tubes. Liquid chromatography was performed using water (1% formic acid) as mobile phase A/C and acetonitrile/methanol (50:50; v:v containing 1% formic acid) as mobile phase B/D. The trapping column used was a Phenomenex, Strata-X, 25 μm, 20×2.0 mm and the analytical column used was a Agilent, Zorbax SB-C8, 3.5 μm, 50×2.1 mm. The column temperature was set to 40° C. and the injection volume was set to 5 or 10 μL. The applied gradient was as described in Table i:

TABLE i

| | Pumps A, B | | | Pumps C, D | | |
|---|---|---|---|---|---|---|
| | Flow | | | Flow | | |
| Time | (mL/min) | A % | B % | (mL/min) | C % | D % |
| Initial | 0.5 | 100 | 0 | 0.3 | 100 | 0 |
| 0.50 | 0.5 | 100 | 0 | 0.3 | 100 | 0 |
| 0.51 | 0.1 | 100 | 0 | 0.3 | 100 | 0 |
| 2.0 | 0.1 | 100 | 0 | 0.3 | | |
| 2.1 | 0.1 | 2 | 98 | 0.3 | | |
| 3.0 | 0.1 | 2 | 98 | 0.3 | 2 | 98 |
| 3.5 | 0.1 | 2 | 98 | 0.3 | 2 | 98 |
| 3.6 | 0.1 | 2 | 98 | 0.5 | 2 | 98 |
| 4.8 | 0.1 | 2 | 98 | 0.5 | 2 | 98 |
| 4.9 | 0.5 | 2 | 98 | 0.5 | 2 | 98 |
| 5.5 | 0.5 | 2 | 98 | 0.5 | 2 | 98 |
| 5.6 | 0.5 | 100 | 0 | 0.3 | 100 | 0 |
| 6.5 | 0.5 | 100 | 0 | 0.3 | 100 | 0 |

From time 0.0 to 0.5 min the analytes were loaded onto the trapping column. At 0.51 min the trapping column was switched in back-flush mode to the analytical column. At 5 min the trapping column was switched back to the load position.

Detection was carried out using a triple-stage quadrupole MS/MS (QTrap 4500: Applied Biosystem, Toronto, Canada) in the selected reaction monitoring mode.

Selected reaction monitoring was as shown in Table ii:

TABLE ii

| | Precursor | Product | Dwell | Declustering | Collision |
|---|---|---|---|---|---|
| | ion | ion | time | potential | energy |
| Analyte | (m/z) | (m/z) | (ms) | (eV) | (eV) |
| Gemcitabine | 264 | 112 | 40 | 76 | 21 |
| BAL27862 | 388 | 106 | 40 | 61 | 69 |
| BAL27862-d7 | 395 | 109 | 40 | 61 | 69 |
| (IS) | | | | | |
| BAL101553 | 516 | 84 | 40 | 96 | 79 |
| BAL101553-d7 | 523 | 84 | 40 | 130 | 73 |
| (IS) | | | | | |

The following calibration ranges in mouse plasma were applied: 1.00 to 500 ng/mL and 10 to 10000 ng/mL for BAL27862 and 10.0 to 10000 ng/mL for BAL101553 and Gemcitabine. Thus, the limit-of-quantitation (LOQ) was: 1 ng/mL for BAL27862 and 10.0 ng/mL for BAL101553 and Gemcitabine.

BAL101553 was used as the dihydrochloride salt and gemcitabine was used as the hydrochloride salt.

### Example 1: Antitumor Activity and Induction of Complete Pathological Tumor Regression in the Patient-Derived Pancreatic Tumor Xenograft Model PAXF1657 after Combined Treatment Modalities with BAL101553 and Gemcitabine

The antitumor effect of the combination treatment with BAL101553 (prodrug of BAL27862) and gemcitabine was evaluated in the established in vivo subcutaneous patient-derived pancreatic mouse xenograft model PAXF1657. By using 3 different dosing regimens of BAL101553 as monotherapy, standard of care (SoC) dosing of gemcitabine monotherapy and the combination treatment of two different dosing regimens of BAL101553 with SoC gemcitabine, the anticancer effect of single agent treatments and of the combined treatments were determined and compared. In each case, animals which presented no measurable tumor burden after the observation period (day 60), and therefore were potentially tumor free, were pathologically assessed for residual tumor cells by an histological approach analyzing the tumor implantation site including surrounding tissue, and if found negative for tumor cells, were declared as cured.

BAL101553 Oral Daily Combined with 240 ml/kg (SoC) Gemcitabine Weekly i.v.

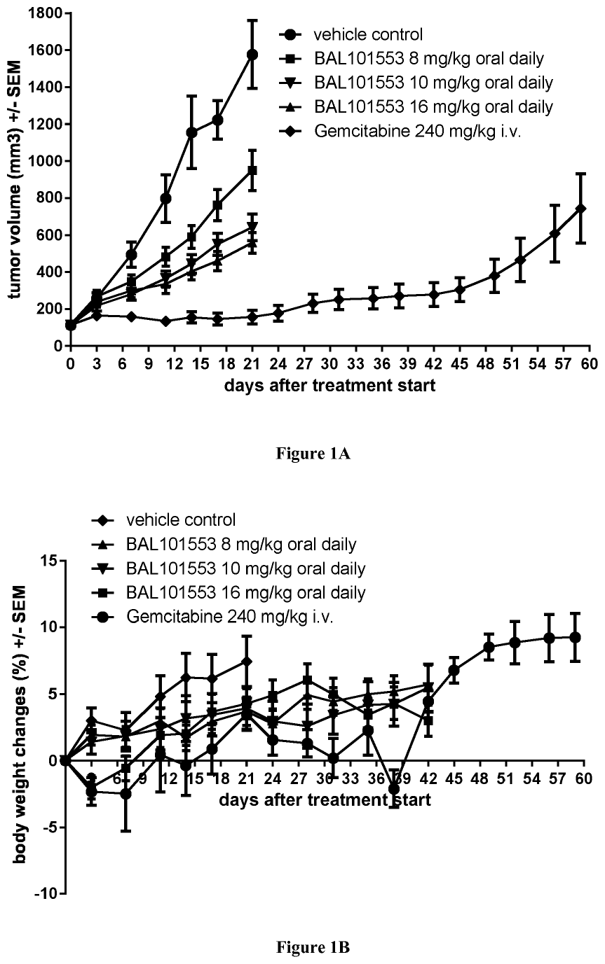

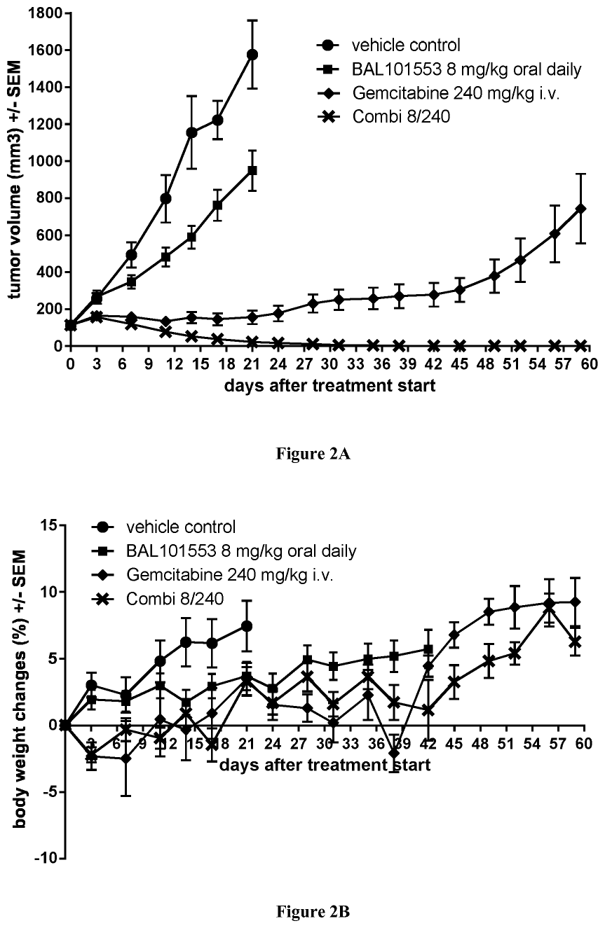

FIGS. 1 to 3 show the antitumor efficacy (−A=mean tumor volume, upper panel) and the body weight changes (−B=mean 0, lower panel) of single agent and combination treatments from day 0 to day 60. Dosing schemes are outlined in Table 1. FIG. 1A shows the vehicle control and all single agent groups whereas FIGS. 2 and 3 show the antitumor activity of the combination treatments as compared to the relevant single agent groups and the vehicle control. Single agent treatments not leading to tumor regressions are depicted until day 21 when the control tumors reached the maximum allowed size, otherwise if regression was observed, the antitumor activity of the treatment groups is shown for the duration of the whole experiment.

Table 2 summarizes the results and shows that 40-80% of the animals treated with the combinations (BAL101553/Gemcitabine: 10/240 or 8/240) were cured. Single agent treatments with BAL101553, even at higher dose (16 n/kg), did neither lead to tumor free animals nor to tumor regression. Gemcitabine monotherapy as SoC for this tumor type, induced regression in some of the animals, but did not lead to tumor free animals (no cures). The single agent treatments and the combinations were well tolerated causing only minimal overall changes in body-weight compared to vehicle-control (Table 3). However one animal in the Gemcitabine monotherapy dosing group had to be euthanized on day 8 due to body weight loss >200. All dosing groups gained weight after the treatment stop on day 42 (FIG. 2B/3).

These results show that BAL1553 combines with Gemcitabine in a synergistic manner leading to tumor free animals or cures (i.e. complete eradication of the tumor burden) compared to single agent treatment.

TABLE 1
Dosing Scheme Example 1

| | Number of | | | |
|---|---|---|---|---|
| Treatment | animals per | Schedule | | |
| groups | group | BAL101553 | Gemcitabine | Vehicle |
| Vehicle control (oral) | 10 | — | — | qd (h:4), from day 18 x 38* |
| 10 ml/kg | | | | |
| BAL101553 | 10 | qd (h:4), from day 18 x 42 | — | — |
| 8 mg/kg (oral) | | | | |
| BAL101553 | 10 | qd (h:4), from day 18 x 42 | — | — |
| 10 mg/kg (oral) | | | | |
| BAL101553 | 10 | qd (h:4), from day 18 x 42 | — | — |
| 16 mg/kg (oral) | | | | |
| Gemcitabine | 10 | — | qw (h:0), from day 18 x 6 | — |
| 240 mg/kg i.v. | | | | |
| Combi 240/8 | 10 | qd (h:4), from day 18 x 42 | qw (h:0), from day 18 x 6 | — |
| Combi 240/10 | 10 | qd (h:4), from day 18 x 42 | qw (h:0), from day 18 x 6 | — |

*All mice in the vehicle control group had to be euthanized at day 38 due to tumor load exceeding the acceptable tumor size. Vehicle BAL0101553: 99.7% (v/v) saline solution (0.9% NaCl), 0.3% (v/v) sodium acetate; vehicle Gemcitabine: 0.9% NaCl. In the combination groups BALI01553 was always gavaged 4 h after the Gemcitabine i.v. application.

TABLE 2
Antitumor Efficacy and Induction of Cures in the patient-derived PAXF1657 pancreatic xenograft model

| | | | | | | Day 60 after first dose | |
|---|---|---|---|---|---|---|---|
| | | | Day 21 after first dose | | | (endpoint) | |
| Treatment and | Dose and | | | # of regressed | # of tumors | # of confirmed | |
| administration | Schedule: | Treatment | | tumors | with | cures | |
| route | D 1-D 42 | until day | T/C | (total) | TV ≤ 4 mm³ (total) | (total) | % cures |
| BAL101553 p.o. | 8 mg/kg qd (h: 4) | 42 | 0.52 | 0 (10) | 0 (20) | 0 (10) | 0 |
| | x42 | | | | | | |
| BAL101553, p.o. | 10 mg/kg qd (h: 4) | 42 | 0.33 | 0 (10) | 0 (10) | 0 (10) | 0 |
| | x42 | | | | | | |
| BAL101553, p.o. | 16 mg/kg qd (h: 4) | 42 | 0.28 | 0 (10) | 0 (10) | 0 (10) | 0 |
| | x42 | | | | | | |
| Gemcitabine, i.v. | 240 mg/kg qw | 42 | 0.03 | 4 (10) | 1 (10) | 0 (10) | 0 |
| | (h: 0) x 6 | | | | | | |
| Gemcitabine, i.v. | 240 mg/kg | 42 | −0.06 | 10 (10) | 2 (10) | 8 (10) | 80 |
| BAL101553, p.o. | 8 mg/kg | | | | | | |
| Gemictabine, i.v | 240 mg/kg | 42 | −0.05 | 10 (10) | 2 (10) | 4 (10) | 40 |
| BAL101553, p.o. | 10 mg/kg | | | | | | |

The vehicle group was dosed orally (p.o.), daily (qd) on days 1-38. BAL101553 was always administered orally (p.o.) and Gemcitabine always i.v. at the doses shown on days 0-42.

TABLE 3
Tolerability in the PAXF1657 pancreatic xenograft model

| Treatment and | Dose and | | Day 21 after first dose | | | |
|---|---|---|---|---|---|---|
| administration | Schedule: | Treatment | | Final body- | Body-weight | |
| route | D 1-D 9; D 12-D 34 | until day | T/C | weight (g) | change (g) | Mortalities |
| BAL101553 p.o. | 8 mg/kg qd | 42 | 0.95 | 26.1 ± 1.5 | 0.9 ± 0.8 | 0/10 |
| | (h: 4) x42 | | | | | |
| BAL101553, p.o. | 10 mg/kg qd | 42 | 0.98 | 27.1 ± 2.0 | 1.0 ± 1.2 | 0/10 |
| | (h: 4) x42 | | | | | |
| BAL101553, p.o. | 16 mg/kg qd | 42 | 0.97 | 26.7 ± 1.4 | 1.1 ± 0.9 | 0/10 |
| | (h: 4) x42 | | | | | |
| Gemcitabine, i.v. | 240 mg/kg qw | 42 | 1.0 | 27.5 ± 2.1 | 0.9 ± 0.8 | 1/10* |
| | (h: 0) x 6 | | | | | |
| Gemcitabine, i.v | 240 mg/kg | 42 | 0.96 | 26.4 ± 2.2 | 0.8 ± 0.8 | 0/10 |
| BAL101553, p.o. | 8 mg/kg | | | | | |
| Gemictabine, i.v | 240 mg/kg | 42 | 0.96 | 26.5 ± 1.1 | 0.3 ± 0.9 | 0/10 |
| BAL101553, p.o. | 10 mg/kg | | | | | |

*1 animal was culled on day 8 because of body-weight loss of more than 20%. Results show the mean ± SD for body-weight, BW (g) and the mean change in body-weight (g) from day-0 until day-21. The TC-BW is calculated from the mean fractional-change in the respective treatment-group divided by the mean fractional-change in the vehicle group. The vehicle group was dosed orally (p.o.), daily (qd) on days 0-38. BAL101553 was always administered orally (p.o.) and Gemcitabine always i.v. at the doses shown. The combinations used the same schedules as those used for the monotherapies.

### Example 2: Determination of the Pharmacokinetic Profile of BAL27862 (Parent Drug of BAL101553), Gemcitabine and the Combined Treatment Thereof in Mouse Plasma

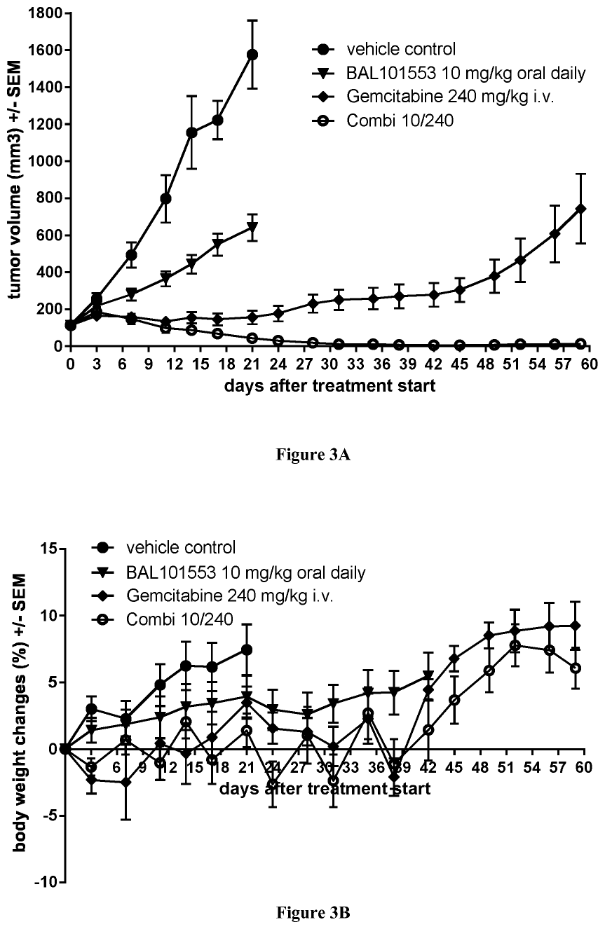

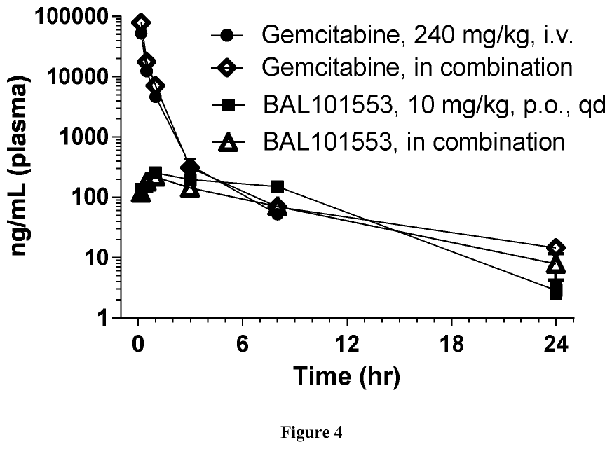

The in vivo combination of BAL101553 with Gemcitabine in the patient-derived PAXF1657 xenograft model led to cures or tumor free animals which was not observed in the single agent treatment groups. In order to rule out drug-drug interactions as an explanation for this observation (e.g. combined treatment may influence the exposure to BAL27862 or Gemcitabine) pharmacokinetic studies in PAXF1657 tumor bearing mice of the same strain were performed. The mice were either treated with BAL101553, Gemcitabine or the combination thereof, and plasma concentrations of BAL27862 and Gemcitabine at various time points were determined (FIG. 4). The analysis showed that the plasma concentrations of either compound did not change significantly if the compounds were administered as single agents or in combination.

This finding indicates that the observed antitumor activity of the combination treatments leading to substantial numbers of cured animals is not due to increased drug exposure as a consequence of any drug-drug interaction, but is rather a consequence of mechanistic interactions on a molecular level.

The following numbered paragraphs describe particular embodiments of the invention.

Paragraph 1. A pharmaceutical combination comprising (a) a compound of formula I

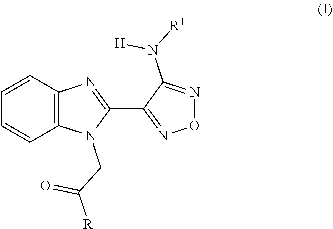

wherein

R represents phenyl or pyridinyl;

wherein phenyl is optionally substituted by one or two substituents independently selected from lower alkyl, lower alkoxy, hydroxyl, amino, lower alkylamino, lower dialkylamino, acetylamino, halogen and nitro;

and wherein pyridinyl is optionally substituted by amino or halogen;

R1 represents hydrogen or cyano-lower alkyl;

and wherein the prefix lower denotes a radical having up to and including a maximum of 4 carbon atoms;

or a pharmaceutically acceptable derivative thereof;

and (b) a compound of formula II (gemcitabine)

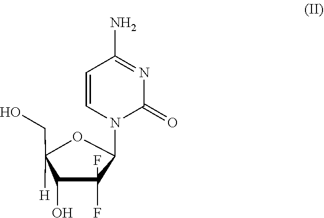

or a pharmaceutically acceptable salt thereof.

Paragraph 2. The pharmaceutical combination according to Paragraph 1, wherein the compound of formula I or pharmaceutically acceptable derivative thereof is a compound of formula I-A

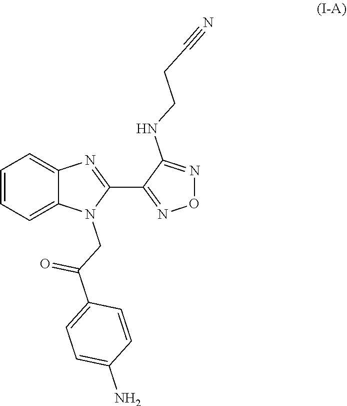 [SMILES: N#CCCNc1nonc1-c1nc2ccccc2n1CC(=O)c1ccc(N)cc1]

or a pharmaceutically acceptable derivative thereof.

Paragraph 3. The pharmaceutical combination according to Paragraph 2, wherein the compound of formula I or pharmaceutically acceptable derivative thereof is the compound of formula I-A or pharmaceutically acceptable salt thereof, or a compound of formula I-B

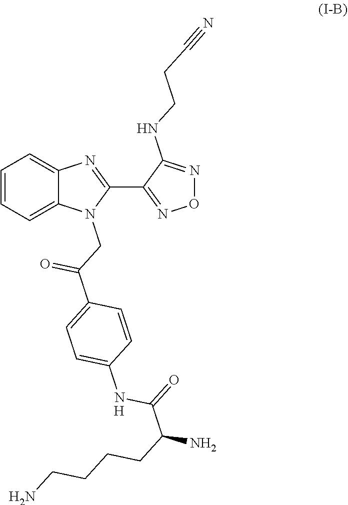

or a pharmaceutically acceptable salt thereof.

Paragraph 4. The pharmaceutical combination according to Paragraph 3, wherein the compound of formula I or pharmaceutically acceptable derivative thereof is the dihydrochloride salt of the compound of formula I-B.

Paragraph 5. The pharmaceutical combination according to any one of Paragraphs 1 to 4, wherein the compound of formula II or a pharmaceutically acceptable salt thereof is gemcitabine hydrochloride.

Paragraph 6. The pharmaceutical combination according to any one of Paragraphs 1 to 5, wherein the compound of formula I or pharmaceutically acceptable derivative thereof and the compound of formula II or pharmaceutically acceptable salt thereof are comprised in separate pharmaceutical compositions.

Paragraph 7. The pharmaceutical combination according to any one of Paragraphs 1 to 6, wherein the mole ratio of the mole amount of the compound of formula II or pharmaceutically acceptable salt thereof to the mole amount of the compound of formula I or pharmaceutically acceptable derivative thereof is 2:1 to 340:1.

Paragraph 8. The pharmaceutical combination according to Paragraph 7, wherein the mole ratio is 5:1 to 95:1.

Paragraph 9. The pharmaceutical combination according to Paragraph 7, wherein the mole ratio is 5:1 to 85:1.

Paragraph 10. A method for treating a neoplastic disease in a subject in need thereof, in particular a human, comprising administering to the subject a therapeutically effective amount of a pharmaceutical combination as defined in any one of Paragraphs 1 to 9.

Paragraph 11. The method according to Paragraph 10, wherein the compound of formula I or pharmaceutically acceptable derivative thereof and the compound of formula II or pharmaceutically acceptable salt thereof are administered simultaneously, sequentially or separately to the subject.

Paragraph 12. The method according to Paragraph 10 or Paragraph 11, wherein the compound of formula I or pharmaceutically acceptable derivative thereof and the compound of formula II or pharmaceutically acceptable salt thereof are administered to the subject according to cyclic treatment schedules;

wherein when the treatment cycles are of the same duration the mole ratio of the total mole amount of the doses of the compound of formula II or pharmaceutically acceptable salt thereof to the total mole amount of the doses of the compound of formula I or pharmaceutically acceptable derivative thereof administered to the subject over the respective treatment cycles is 2:1 to 340:1;

and wherein when the treatment cycles are of different duration the ratio of the total mole amount of the doses of the compound of formula II or pharmaceutically acceptable salt thereof to the total mole amount of the doses of the compound of formula I or pharmaceutically acceptable derivative thereof administered to the subject over a theoretical period of time corresponding to a common multiple of the duration of the respective treatment cycles is 2:1 to 340:1.

Paragraph 13. The method according to Paragraph 12, wherein the mole ratio is 5:1 to 95:1.

Paragraph 14. The method according to Paragraph 12, wherein the mole ratio is 5:1 to 85:1.

Paragraph 15. The method according to Paragraph 10 or Paragraph 11, wherein the compound of formula I or pharmaceutically acceptable derivative thereof is administered to the subject according to a continuous treatment schedule and the compound of formula II or pharmaceutically acceptable salt thereof is administered to the subject according to a cyclic treatment schedule and wherein the mole ratio of the total mole amount of the doses of the compound of formula II or pharmaceutically acceptable salt thereof administered to the subject over a treatment cycle to the mole amount of the doses of the compound of formula I or derivative thereof over a period of the same duration of the treatment cycle of the compound of formula II or pharmaceutically acceptable salt thereof is 2:1 to 340:1.

Paragraph 16. The method according to Paragraph 15, wherein the mole ratio is 5:1 to 95:1.

Paragraph 17. The method according to Paragraph 15, wherein the mole ratio is 5:1 to 85:1.

Paragraph 18. The method according to any one of Paragraphs 10 to 17, wherein the compound of formula I or pharmaceutically acceptable derivative thereof is administered orally at dose corresponding to the mole equivalent of about 2 mg to about 30 mg of the dihydrochloride salt of the compound of formula I-B per day on days when administered, and wherein the compound of formula II or pharmaceutically acceptable salt thereof is administered intravenously at a dose corresponding to the mole equivalent of Gemcitabine hydrochloride of about 100 mg/m² to 1400 mg/m² per week during weeks when administered.

Paragraph 19. The method according to Paragraph 18, wherein the compound of formula I or pharmaceutically acceptable derivative thereof is administered at a dose corresponding to the mole equivalent of about 4 mg to about 20 mg of the dihydrochloride salt of the compound of formula I-B per day on days when administered.

Paragraph 20. The method according to Paragraph 18, wherein the compound of formula I or pharmaceutically acceptable derivative thereof is administered at a dose corresponding to the mole equivalent of about 8 mg to about 20 mg of the dihydrochloride salt of the compound of formula I-B per day on days when administered.

Paragraph 21. The method according to any one of Paragraph 18 to 20, wherein the compound of formula I or pharmaceutically acceptable derivative thereof is administered to the subject at least once per day.

Paragraph 22. The method according to any one of Paragraphs 18 to 21, wherein the compound of formula II or pharmaceutically acceptable salt thereof is administered at a dose corresponding to the mole equivalent of Gemcitabine hydrochloride of about 200 mg/m² to about 1300 mg/m² per week during weeks when administered.

Paragraph 23. The method according to any one of Paragraphs 18 to 22, wherein the compound of formula II or pharmaceutically acceptable salt thereof is administered to the subject according to a 21-day treatment cycle with administration initiated on days 1 and 8, or according to a 28-day treatment cycle with administration initiated on days 1, 8 and 15.

Paragraph 24. The method according to any one of Paragraphs 10 to 17, wherein the compound of formula I or pharmaceutically acceptable derivative thereof is administered intravenously at a dose corresponding to the mole equivalent of about 15 mg/m² to about 160 mg/m² of the dihydrochloride salt of the compound of formula I-B per week during weeks when administered, and wherein the compound of formula II or pharmaceutically acceptable salt thereof is administered intravenously at a dose corresponding to the mole equivalent of Gemcitabine hydrochloride of about 100 mg/m² to 1400 mg/m² per week during weeks when administered.

Paragraph 25. The method according to Paragraph 24, wherein the compound of formula I or pharmaceutically acceptable derivative thereof is administered at a dose corresponding to the mole equivalent of about 30 mg/m² to about 100 mg/m² of the dihydrochloride salt of the compound of formula I-B per week during weeks when administered.

Paragraph 26. The method according to Paragraph 24, wherein the compound of formula I or pharmaceutically acceptable derivative thereof is administered at a dose corresponding to the mole equivalent of about 30 mg/m² to about 70 mg/m² of the dihydrochloride salt of the compound of formula I-B per week during weeks when administered.

Paragraph 27. The method according to any one of Paragraphs 24 to 26, wherein the compound of formula I or pharmaceutically acceptable derivative thereof is administered according to a 21-day treatment cycle with administration initiated on days 1 and 8, or a 28-day treatment cycle with administration initiated on days 1, 8 and 15.

Paragraph 28. The method according to any one of Paragraphs 24 to 27, wherein the compound of formula II or pharmaceutically acceptable salt thereof is administered intravenously at a dose corresponding to the mole equivalent of gemcitabine hydrochloride of about 200 mg/m² to about 1300 mg/m² per week during weeks when administered.

Paragraph 29. The method according to any one of Paragraphs 24 to 28, wherein the compound of formula II or pharmaceutically acceptable salt thereof is administered according to a 21-day treatment cycle with administration initiated on days 1 and 8, or according to a 28-day treatment cycle with administration initiated on days 1, 8 and 15.

Paragraph 30. The method according to any one of Paragraphs 10 to 29, wherein the neoplastic disease is a solid tumour.

Paragraph 31. The method according to any one of Paragraphs 10 to 30, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous neoplasms, meningiomas, nerve sheath tumours, granular cell tumours and alveolar soft part sarcomas, Hodgkin's and non-Hodgkin's lymphomas, other lymphoreticular neoplasms, plasma cell tumours, mast cell tumours, immunoproliferative diseases, leukemias, myeloproliferative disorders, lymphoproliferative disorders and myelodysplastic syndromes.

Paragraph 32. The method according to any one of Paragraphs 10 to 31, wherein the disease is a cancer.

Paragraph 33. The method according to Paragraph 32, wherein the cancer in terms of the organs and parts of the body affected is selected from the brain, breast (including triple negative breast cancer), cervix, ovaries, colon, rectum, (including colon and rectum i.e. colorectal cancer), lung, (including small cell lung cancer, non-small cell lung cancer, large cell lung cancer and mesothelioma), endocrine system, bone, adrenal gland, thymus, liver, stomach, intestine, (including gastric cancer), pancreas, bone marrow, haematological malignancies, (such as lymphoma, leukaemia, myeloma or lymphoid malignancies), bile duct, bladder, urinary tract, kidneys, skin, thyroid, head, neck, prostate and testis.

Paragraph 34. The method according to Paragraph 32, wherein the cancer is selected from brain cancer (e.g. glioblastoma), breast cancer (including triple negative breast cancer), prostate cancer, cervical cancer, ovarian cancer, biliary cancer, gastric cancer, colorectal cancer, pancreatic cancer, liver cancer, brain cancer, neuroendocrine cancer, lung cancer, kidney cancer, haematological malignancies, melanoma and sarcomas.

Paragraph 35. The method according to Paragraph 34, wherein the cancer is a cancer selected from lung cancer (including non-small cell lung cancer and mesothelioma), pancreatic cancer (including ductal adenocarcinoma and metastatic pancreatic cancer), bladder cancer, ovarian cancer (including ovarian carcinoma), biliary tract cancer, peripheral or cutaneous T-cell lymphoma, non-Hodgkins lymphoma and breast cancer, in particular pancreatic cancer.

Paragraph 36. A method for treating a neoplastic disease in a subject in need thereof, in particular a human, comprising administering to the subject a therapeutically effective amount of a compound of formula I or a pharmaceutically acceptable derivative thereof as defined in any one of Paragraphs 1 to 4, which subject is undergoing or will undergo treatment with a compound of formula II or pharmaceutically acceptable salt thereof as defined in Paragraph 1 or Paragraph 5.

Paragraph 37. The method according to Paragraph 36, wherein the compound of formula I or a pharmaceutically acceptable derivative thereof is administered to the subject as defined in any one of Paragraphs 11 to 29.

Paragraph 38. The method according to Paragraph 34 or Paragraph 35, wherein the neoplastic disease is as defined in any one of Paragraphs 30 to 35.

Paragraph 39. A method for treating a neoplastic disease in a subject in need thereof, in particular a human, comprising administering to the subject a therapeutically effective amount of a compound of formula II or a pharmaceutically acceptable salt thereof as defined in Paragraph 1 or Paragraph 5, which subject is undergoing or will undergo treatment with a compound of formula I or pharmaceutically acceptable derivative thereof as defined in any one of Paragraphs 1 to 4.

Paragraph 40. The method according to Paragraph 39, wherein the compound of formula II or a pharmaceutically acceptable salt thereof is administered to the subject as defined in any one of Paragraphs 11 to 29.

Paragraph 41. The method according to Paragraph 34 or Paragraph 35, wherein the neoplastic disease is as defined in any one of Paragraphs 30 to 35.

Paragraph 42. A pharmaceutical combination as defined in any one of Paragraphs 1 to 9, for use in the treatment of a neoplastic disease in a subject, in particular a human.

Paragraph 43. The pharmaceutical combination for use according to Paragraph 42, wherein the compound of formula I or pharmaceutically acceptable derivative thereof and the compound of formula II or pharmaceutically acceptable salt thereof are for simultaneous, sequential or separate administration to the subject.

Paragraph 44. The pharmaceutical combination for use according to Paragraph 42 or Paragraph 43, wherein the compound of formula I or pharmaceutically acceptable derivative thereof and the compound of formula II or pharmaceutically acceptable salt thereof are administered to the subject according to cyclic treatment schedules;

wherein when the treatment cycles are of the same duration the mole ratio of the total mole amount of the doses of the compound of formula II or pharmaceutically acceptable salt thereof to the total mole amount of the doses of the compound of formula I or pharmaceutically acceptable derivative thereof administered to the subject over the respective treatment cycles is 2:1 to 340:1;

and wherein when the treatment cycles are of different duration the ratio of the total mole amount of the doses of the compound of formula II or pharmaceutically acceptable salt thereof to the total mole amount of the doses of the compound of formula I or pharmaceutically acceptable derivative thereof administered to the subject over a theoretical period of time corresponding to a common multiple of the duration of the respective treatment cycles is 2:1 to 340:1.

Paragraph 45. The pharmaceutical combination for use according to Paragraph 44, wherein the mole ratio is 5:1 to 95:1.

Paragraph 46. The pharmaceutical combination for use according to Paragraph 44, wherein the mole ratio is 5:1 to 85:1.

Paragraph 47. The pharmaceutical combination for use according to Paragraph 42 or Paragraph 43, wherein the compound of formula I or pharmaceutically acceptable derivative thereof is administered to the subject according to a continuous treatment schedule and the compound of formula II or pharmaceutically acceptable salt thereof is administered to the subject according to a cyclic treatment schedule and wherein the mole ratio of the total mole amount of the doses of the compound of formula II or pharmaceutically acceptable salt thereof administered to the subject over a treatment cycle to the mole amount of the doses of the compound of formula I or derivative thereof over a period of the same duration of the treatment cycle of the compound of formula II or pharmaceutically acceptable salt is 2:1 to 340:1.

Paragraph 48. The pharmaceutical combination for use according to Paragraph 47, wherein the mole ratio is 5:1 to 95:1.

Paragraph 49. The pharmaceutical combination for use according to Paragraph 47, wherein the mole ratio is 5:1 to 85:1.

Paragraph 50. The pharmaceutical combination for use according to any one of Paragraphs 42 to 49, wherein the compound of formula I or pharmaceutically acceptable derivative thereof is administered orally at dose corresponding to the mole equivalent of about 2 mg to about 30 mg of the dihydrochloride salt of the compound of formula I-B per day on days when administered, and wherein the compound of formula II or pharmaceutically acceptable salt thereof is administered intravenously at a dose corresponding to the mole equivalent of gemcitabine hydrochloride of about 100 mg/m² to 1400 mg/m² per week during weeks when administered.

Paragraph 51. The pharmaceutical combination for use according to Paragraph 50, wherein the compound of formula I or pharmaceutically acceptable derivative thereof is administered at a dose corresponding to the mole equivalent of about 4 mg to about 20 mg of the dihydrochloride salt of the compound of formula I-B per day on days when administered.

Paragraph 52. The pharmaceutical combination for use according to Paragraph 50, wherein the compound of formula I or pharmaceutically acceptable derivative thereof is administered at a dose corresponding to the mole equivalent of about 8 mg to about 20 mg of the dihydrochloride salt of the compound of formula I-B per day on days when administered.

Paragraph 53. The pharmaceutical combination for use according to any one of Paragraphs 50 to 52, wherein the compound of formula I or pharmaceutically acceptable derivative thereof is administered to the subject at least once per day.

Paragraph 54. The pharmaceutical combination for use according to any one of Paragraphs 50 to 53, wherein the compound of formula II or pharmaceutically acceptable salt thereof is administered at a dose corresponding to the mole equivalent of gemcitabine hydrochloride of about 200 mg/m² to about 1300 mg/m² per week during weeks when administered.

Paragraph 55. The pharmaceutical combination for use according to any one of Paragraphs 50 to 54, wherein the compound of formula II or pharmaceutically acceptable salt thereof is administered to the subject according to a 21-day treatment cycle with administration initiated on days 1 and 8, or according to a 28-day treatment cycle with administration initiated on days 1, 8 and 15.

Paragraph 56. The pharmaceutical combination for use according to any one of Paragraphs 42 to 49, wherein the compound of formula I or pharmaceutically acceptable derivative thereof is administered intravenously at a dose corresponding to the mole equivalent of about 15 mg/m² to about 160 mg/m² of the dihydrochloride salt of the compound of formula I-B per week during weeks when administered, and wherein the compound of formula II or pharmaceutically acceptable salt thereof is administered intravenously at a dose corresponding to the mole equivalent of gemcitabine hydrochloride of about 100 mg/m² to 1400 mg/m² per week during weeks when administered.

Paragraph 57. The pharmaceutical combination for use according to Paragraph 56, wherein the compound of formula I or pharmaceutically acceptable derivative thereof is administered at a dose corresponding to the mole equivalent of about 30 mg/m² to about 100 mg/m² of the dihydrochloride salt of the compound of formula I-B per week during weeks when administered.

Paragraph 58. The pharmaceutical combination for use according to Paragraph 56, wherein the compound of formula I or pharmaceutically acceptable derivative thereof is administered at a dose corresponding to the mole equivalent of about 30 mg/m² to about 70 mg/m² of the dihydrochloride salt of the compound of formula I-B per week during weeks when administered.

Paragraph 59. The pharmaceutical combination for use according to any one of Paragraphs 56 to 58, wherein the compound of formula I or pharmaceutically acceptable derivative thereof is administered according to a 21-day treatment cycle with administration initiated on days 1 and 8, or a 28-day treatment cycle with administration initiated on days 1, 8 and 15.

Paragraph 60. The pharmaceutical combination for use according to any one of Paragraphs 56 to 59, wherein the compound of formula II or pharmaceutically acceptable salt thereof is administered intravenously at a dose corresponding to the mole equivalent of gemcitabine hydrochloride of about 200 mg/m² to about 1300 mg/m² per week during weeks when administered.

Paragraph 61. The pharmaceutical combination for use according to any one of Paragraphs 56 to 59, wherein the compound of formula II or pharmaceutically acceptable salt thereof is administered according to a 21-day treatment cycle with administration initiated on days 1 and 8, or according to a 28-day treatment cycle with administration initiated on days 1, 8 and 15.

Paragraph 62. The pharmaceutical combination for use according to any one of Paragraphs 42 to 61, wherein the neoplastic disease is a solid tumour.

Paragraph 63. The pharmaceutical combination for use according to any one of Paragraphs 42 to 61, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous neoplasms, meningiomas, nerve sheath tumours, granular cell tumours and alveolar soft part sarcomas, Hodgkin's and non-Hodgkin's lymphomas, other lymphoreticular neoplasms, plasma cell tumours, mast cell tumours, immunoproliferative diseases, leukemias, myeloproliferative disorders, lymphoproliferative disorders and myelodysplastic syndromes.

Paragraph 64. The pharmaceutical combination for use according to any one of Paragraphs 42 to 63, wherein the disease is a cancer.

Paragraph 65. The pharmaceutical combination for use according to Paragraph 64, wherein the cancer in terms of the organs and parts of the body affected is selected from the brain, breast (including triple negative breast cancer), cervix, ovaries, colon, rectum, (including colon and rectum i.e. colorectal cancer), lung, (including small cell lung cancer, non-small cell lung cancer, large cell lung cancer and mesothelioma), endocrine system, bone, adrenal gland, thymus, liver, stomach, intestine, (including gastric cancer), pancreas, bone marrow, haematological malignancies, (such as lymphoma, leukaemia, myeloma or lymphoid malignancies), bile duct, bladder, urinary tract, kidneys, skin, thyroid, head, neck, prostate and testis.

Paragraph 66. The pharmaceutical combination for use according to Paragraph 64, wherein the cancer is selected from brain cancer (e.g. glioblastoma), breast cancer (including triple negative breast cancer), prostate cancer, cervical cancer, ovarian cancer, biliary cancer, gastric cancer, colorectal cancer, pancreatic cancer, liver cancer, brain cancer, neuroendocrine cancer, lung cancer, kidney cancer, haematological malignancies, melanoma and sarcomas.

Paragraph 67. The pharmaceutical combination for use according to Paragraph 66, wherein the cancer is a cancer selected from lung cancer (including non-small cell lung cancer and mesothelioma), pancreatic cancer (including ductal adenocarcinoma and metastatic pancreatic cancer), bladder cancer, ovarian cancer (including ovarian carcinoma), biliary tract cancer, peripheral or cutaneous T-cell lymphoma, non-Hodgkins lymphoma and breast cancer, in particular pancreatic cancer.

Paragraph 68. A compound of formula I or a pharmaceutically acceptable derivative thereof as defined in any one of Paragraphs 1 to 4, for use in combination with a compound of formula II or pharmaceutically acceptable salt thereof as defined in Paragraph 1 or Paragraph 5, for the treatment of a neoplastic disease in a subject, in particular a human.

Paragraph 69. The compound of formula I or a pharmaceutically acceptable derivative thereof for use according to Paragraph 64, wherein the compound of formula I or a pharmaceutically acceptable derivative thereof is administered to the subject as defined in any one of Paragraphs 42 to 61. Paragraph 70. The compound of formula I or a pharmaceutically acceptable derivative thereof for use according to Paragraph 68 or Paragraph 69, wherein the neoplastic disease is as defined in any one of Paragraphs 62 to 67.

Paragraph 71. A compound of formula II or pharmaceutically acceptable salt thereof as defined in Paragraph 1 or Paragraph 5, for use in combination with a compound of formula I or pharmaceutically acceptable derivative thereof as defined in any one of Paragraphs 1 to 4, for the treatment of a neoplastic disease.

Paragraph 72. The compound of formula II or pharmaceutically acceptable salt thereof for use according to Paragraph 71, wherein the compound of formula II or a pharmaceutically acceptable salt thereof is administered to the subject as defined in any one of Paragraphs 42 to 61.

Paragraph 73. The compound of formula II or pharmaceutically acceptable salt thereof for use according to Paragraph 71 or Paragraph 72, wherein the neoplastic disease is as defined in any one of Paragraphs 62 to 67.

Paragraph 74. Use of a pharmaceutical combination as defined in any one of Paragraphs 1 to 5 in the preparation of single-agent medicaments or as a combined medicament for the treatment of a neoplastic disease in a subject, in particular a human.

Paragraph 75. Use according to Paragraph 74, wherein the compound of formula I or pharmaceutically acceptable derivative thereof and the compound of formula II or pharmaceutically acceptable salt thereof are for simultaneous, sequential or separate administration to the subject.

Paragraph 76. Use according to Paragraph 74 or Paragraph 75, wherein the compound of formula I or pharmaceutically acceptable derivative thereof and the compound of formula II or a pharmaceutically acceptable salt thereof are administered to the subject as defined in any one of Paragraphs 42 to 61.

Paragraph 77. Use according to any one of Paragraphs 74 to 76, wherein the neoplastic disease is as defined in any one of Paragraphs 62 to 67.

Paragraph 78. Use of a compound of formula I or a pharmaceutically acceptable derivative thereof as defined in any one of Paragraphs 1 to 4 in the preparation of a single-agent medicament for use in combination with a compound of formula II or pharmaceutically acceptable salt thereof or in the preparation of a combined medicament with the compound of formula II or pharmaceutically acceptable salt thereof, for the treatment of a neoplastic disease in a subject, in particular a human.

Paragraph 79. Use according to Paragraph 78, wherein the compound of formula I or pharmaceutically acceptable derivative thereof is administered to the subject as defined in any one of Paragraphs 42 to 61.

Paragraph 80. Use according to Paragraph 74 or Paragraph 75, wherein the neoplastic disease is as defined in any one of Paragraphs 62 to 67.

Paragraph 81. Use of a compound of formula II or a pharmaceutically acceptable salt thereof as defined in Paragraph 1 or Paragraph 5 in the preparation of a single-agent medicament for use in combination with a compound of formula I or pharmaceutically acceptable derivative thereof or in the preparation of a combined medicament with the compound of formula I or pharmaceutically acceptable derivative thereof, for the treatment of a neoplastic disease in a subject, in particular a human.

Paragraph 82. Use according to Paragraph 81, wherein the compound of formula II or pharmaceutically acceptable salt thereof is administered to the subject as defined in any one of Paragraphs 42 to 61.

Paragraph 83. Use according to Paragraph 81 or Paragraph 82, wherein the neoplastic disease is as defined in any one of Paragraphs 62 to 67.

Paragraph 84. A kit comprising a pharmaceutical combination as defined in any one of Paragraphs 1 to 9, wherein component (a) and component (b) are provided as separate dosage units.

Paragraph 85. The kit according to Paragraph 84, wherein the kit is for use in treating a neoplastic disease.

Paragraph 86. The kit according to paragraph 85, further comprising instructions for simultaneous, separate or sequential administration thereof for use in the treatment of a neoplastic disease, in particular a cancer, in a subject, in particular a human.

1. A pharmaceutical combination, comprising (a) a compound of formula I: 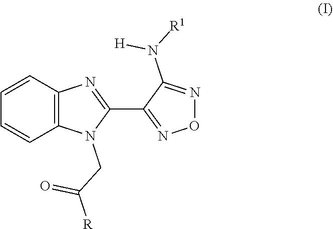
wherein:
R represents phenyl or pyridinyl;
wherein phenyl is optionally substituted by one or two substituents independently selected from lower alkyl, lower alkoxy, hydroxyl, amino, lower alkylamino, lower dialkylamino, acetylamino, halogen and nitro;
and wherein pyridinyl is optionally substituted by amino or halogen;
R1 represents hydrogen or cyano-lower alkyl;
and wherein the prefix lower denotes a radical having up to and including a maximum of 4 carbon atoms; or a pharmaceutically acceptable derivative thereof,
and (b) a compound of formula II (gemcitabine) 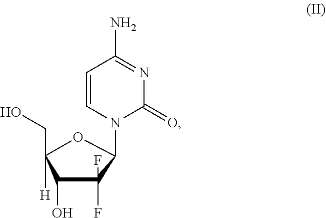
or a pharmaceutically acceptable salt thereof.

2. The pharmaceutical combination according to claim 1, wherein the compound of formula I, or pharmaceutically acceptable derivative thereof, is a compound of formula I-A: 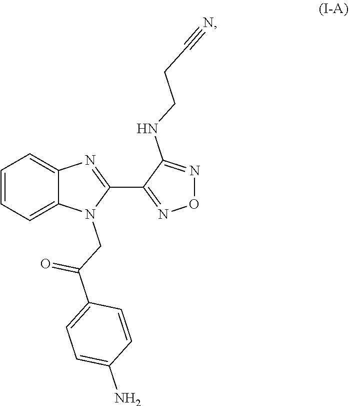 [SMILES: N#CCCNc1nonc1-c1nc2ccccc2n1CC(=O)c1ccc(N)cc1]
or a pharmaceutically acceptable salt thereof, or a compound of formula I-B: 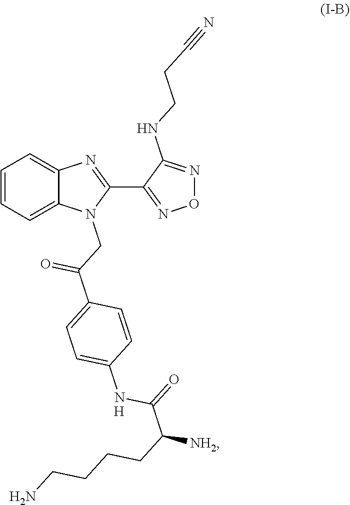
or a pharmaceutically acceptable salt thereof.

3. The pharmaceutical combination according to claim 2, wherein the compound of formula I or pharmaceutically acceptable derivative thereof is the dihydrochloride salt of the compound of formula I-B and wherein the compound of formula II or a pharmaceutically acceptable salt thereof is gemcitabine hydrochloride.

4. The pharmaceutical combination according to claim 1, wherein the mole ratio of the mole amount of the compound of formula II or pharmaceutically acceptable salt thereof to the mole amount of the compound of formula II or pharmaceutically acceptable derivative thereof is 2:1 to 340:1.

5. A method for the treatment of a neoplastic disease in a subject in need thereof, comprising the step of administering the pharmaceutical combination according to claim 1 to said subject.

6. The method according to claim 5, wherein the compound of formula I or pharmaceutically acceptable derivative thereof and of the compound of formula II or pharmaceutically acceptable salt thereof are administered to the subject according to cyclic treatment schedules;
wherein when the treatment cycles are of the same duration the mole ratio of the total mole amount of the doses of the compound of formula II or pharmaceutically acceptable salt thereof to the total mole amount of the doses of the compound of formula I or pharmaceutically acceptable derivative thereof administered to the subject over the respective treatment cycles is 2:1 to 340:1;
and wherein when the treatment cycles are of different duration the ratio of the total mole amount of the doses of the compound of formula II or pharmaceutically acceptable salt thereof to the total mole amount of the doses of the compound of formula II or pharmaceutically acceptable derivative thereof administered to the subject over a theoretical period of time corresponding to a common multiple of the duration of the respective treatment cycles is 2:1 to 340:1.

7. The method according to claim 5, wherein the compound of formula I or pharmaceutically acceptable derivative thereof is administered to the subject according to a continuous treatment schedule and the compound of formula II or pharmaceutically acceptable salt thereof is administered to the subject according to a cyclic treatment schedule and wherein the mole ratio of the total mole amount of the doses of the compound of formula II or pharmaceutically acceptable salt thereof administered to the subject over a treatment cycle to the mole amount of the doses of the compound of formula I or derivative thereof over a period of the same duration of the treatment cycle of the compound of formula II or pharmaceutically acceptable salt thereof is 2:1 to 340:1.

8. The method according to claim 5, wherein the compound of formula I or pharmaceutically acceptable derivative thereof is administered orally at dose corresponding to the mole equivalent of about 2 mg to about 30 mg of the dihydrochloride salt of the compound of formula I-B per day on days when administered, and wherein the compound of formula II or pharmaceutically acceptable salt thereof is administered intravenously at a dose corresponding to the mole equivalent of Gemcitabine hydrochloride of 100 mg/m² to 1400 mg/m² per week during weeks when administered.

9. The method according to claim 8, wherein the compound of formula I or pharmaceutically acceptable derivative thereof is administered to the subject at least once per day, and wherein the compound of formula II or pharmaceutically acceptable salt thereof is administered to the subject according to a 21-day treatment cycle with administration initiated on days 1 and 8, or according to a 28-day treatment cycle with administration initiated on days 1, 8 and 15.

10. The method according to claim 5, wherein the compound of formula I or pharmaceutically acceptable derivative thereof is administered intravenously at a dose corresponding to the mole equivalent of about 15 mg/m² to about 160 mg/m² of the dihydrochloride salt of the compound of formula I-B per week during weeks when administered, and wherein the compound of formula II or pharmaceutically acceptable salt thereof is administered intravenously at a dose corresponding to the mole equivalent of gemcitabine hydrochloride of about 100 mg/m² to 1400 mg/m² per week during weeks when administered.

11. The method according to claim 10, wherein the compound of formula I or pharmaceutically acceptable derivative thereof is administered according to a 21-day treatment cycle with administration initiated on days 1 and 8, or a 28-day treatment cycle with administration initiated on days 1, 8 and 15, and wherein the compound of formula II or pharmaceutically acceptable salt thereof is administered according to a 21-day treatment cycle with administration initiated on days 1 and 8, or according to a 28-day treatment cycle with administration initiated on days 1, 8 and 15.

12. The method according to claim 5, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous neoplasms, meningiomas, nerve sheath tumours, granular cell tumours and alveolar soft part sarcomas, Hodgkin's and non-Hodgkin's lymphomas, other lymphoreticular neoplasms, plasma cell tumours, mast cell tumours, immunoproliferative diseases, leukemias, myeloproliferative disorders, lymphoproliferative disorders and myelodysplastic syndromes.

13. The method according to claim 5, wherein the disease is a cancer.

14. A method for the treatment of a neoplastic disease in a subject in need thereof, comprising the step of administering a compound of formula I, or a pharmaceutically acceptable derivative thereof, as defined in claim 1 in combination with a compound of formula II, or pharmaceutically acceptable salt thereof, as defined in claim 1.

15. A method for the treatment of a neoplastic disease in a subject in need thereof, comprising the step of administering to said subject a compound of formula II, or pharmaceutically acceptable salt thereof, as defined in claim 1 in combination with a compound of formula I, or pharmaceutically acceptable derivative thereof, as defined in claim 1.

16. The method according to claim 5, wherein said subject is a human.

17. The method according to claim 13, wherein said cancer is selected from lung cancer (including non-small cell lung cancer and mesothelioma), pancreatic cancer (including ductal adenocarcinoma and metastatic pancreatic cancer), bladder cancer, ovarian cancer (including ovarian carcinoma), biliary tract cancer, peripheral or cutaneous T-cell lymphoma, non-Hodgkins lymphoma and breast cancer.

18. The method according to claim 13, wherein said cancer is pancreatic cancer.

19. The method according to claim 14, wherein said subject is a human.

20. The method according to claim 15, wherein said subject is a human.

21. The method according to claim 15, wherein the neoplastic disease is a cancer selected from lung cancer (including non-small cell lung cancer and mesothelioma), pancreatic cancer (including ductal adenocarcinoma and metastatic pancreatic cancer), bladder cancer, ovarian cancer (including ovarian carcinoma), biliary tract cancer, peripheral or cutaneous T-cell lymphoma, non-Hodgkins lymphoma and breast cancer.

22. The method according to claim 15, wherein the neoplastic disease is pancreatic cancer.
